SEQUENCE LISTING (24 sequences)

SEQ ID NO: 1 - protein, 5 aa, synthetic construct
NDAIN

SEQ ID NO: 2 - protein, 17 aa, synthetic construct
GIFPMFGTAKYSQNFQG

SEQ ID NO: 3 - protein, 14 aa, synthetic construct
SRDPLLYPHHALSP

SEQ ID NO: 4 - protein, 11 aa, synthetic construct
QGDSLRYYYAS

SEQ ID NO: 5 - protein, 7 aa, synthetic construct
GKNNRPS

SEQ ID NO: 6 - protein, 11 aa, synthetic construct
SSRDSSGNNWV

SEQ ID NO: 7 - protein, 123 aa, synthetic construct
QVQLQQSGAEVKKPGSSVRVSCKASGGTFNNDAINWVRQAPGQGLEWMGGIFPMFGTAKYSQNFQGRVAITADESTGTASMELSSLRSEDTAVYYCARSRDPLLYPHHALSPWGRGTMVTVSS

SEQ ID NO: 8 - protein, 108 aa, synthetic construct
SSELTQDPAVSVALGQTVRVTCQGDSLRYYYASWYQQKPGQAPVLVIYGKNNRPSGIPDRFSGSSSGNTASLTITGAQAEDEADYYCSSRDSSGNNWVFGGGTELTVL

SEQ ID NO: 9 - protein, 453 aa, synthetic construct
QVQLQQSGAEVKKPGSSVRVSCKASGGTFNNDAINWVRQAPGQGLEWMGGIFPMFGTAKYSQNFQGRVAITADESTGTASMELSSLRSEDTAVYYCARSRDPLLYPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
SSELTQDPAVSVALGQTVRVTCQGDSLRYYYASWYQQKPGQAPVLVIYGKNNRPSGIPDRFSGSSSGNTASLTITGAQAEDEADYYCSSRDSSGNNWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - DNA, 1359 bp, synthetic construct
  source 1..1359: mol_type = other DNA
caagtacagttgcagcagtccggagcggaagtcaagaagccgggctccagcgtgcgcgtgtcctgtaaagccagcggcggcaccttcaacaacgatgccattaactgggtccgccaggccccgggacagggtcttgaatggatgggcgggatcttcccgatgttcggaactgcaaagtacagccaaaacttccagggccgggtggccatcacagctgacgagtcaaccggaaccgcctccatggagctctcttcgctccgctcggaagatactgccgtgtattactgcgcccggtcacgggaccccctcctgtacccacaccatgctctgtccccctggggcagagggaccatggtcactgtgtcctcggcatccactaaggggcctagcgtctttccgctggccccgtcctccaagtccacttcgggtggaaccgcggcactggggtgcctcgtgaaggactacttccccgagccggtcaccgtgtcctggaactcgggagccctgacctccggagtgcatactttccctgcggtgctgcagtcctccgggctctactcgctgtcaagcgtggtcaccgtcccgagctcatccctgggtactcagacctacatttgcaacgtgaaccacaaaccttccaacaccaaggtcgacaagaaagtggagcctaagagctgcgacaagacccacacctgtcccccgtgtcccgcccctgagctgctgggcggccccagcgtgttcctcttcccgcctaagccgaaggacactctgtacatcaccagagagcctgaagtgacctgtgtggtggtggatgtgtcccacgaggatccggaagtgaagttcaattggtacgtggacggagtggaagtccataacgccaagaccaagccccgcgaggaacagtacaactcaacttaccgggtggtgtcagtgctgaccgtgctgcaccaagattggctgaacgggaaggagtacaagtgcaaagtctccaacaaggcgctgccggcccccattgaaaagaccatcagcaaggctaagggccagccccgggaaccacaggtctacaccttgcccccttcccgggacgaactgaccaagaaccaagtgtcgctgacgtgcctggtcaagggcttttatccatctgacatcgccgtggagtgggaaagcaacggccagccggaaaacaactacaagactaccccgcctgtgctggactccgacggctcgttcttcctgtattccaagctcaccgtggataagtccagatggcagcagggcaatgtgttcagctgcagcgtgatgcatgaggccctgcacaaccactacactcagaaatcactgtccctttcccccggaaaa

SEQ ID NO: 12 - DNA, 642 bp, synthetic construct
  source 1..642: mol_type = other DNA
tcgtccgaactgacccaggacccggctgtgtccgtggccctgggacaaactgtgcgggtcacgtgccagggcgacagcctgcgctactactacgcgtcgtggtaccagcaaaagcctgggcaggcgccggtgttggtgatctacggaaagaacaatcgcccctccggaattccagacaggttcagcggcagctcatccgggaacaccgcctcactgaccatcaccggagcccaggccgaagatgaagctgactactactgctcctcgagagattccagcggcaacaactgggtgtttggtggcggaacagagctcactgtgctcgggcagccgaaggctgcgccctcggtgacccttttcccgccatcatccgaggagctgcaggccaacaaggccacgctcgtgtgcctgatctcggatttctaccctggtgccgtgactgtggcctggaaggcagactctagccccgtgaaggccggagtggagactaccaccccgtccaagcagtccaacaacaaatacgccgcgtcatcctacctgtccctgacccctgaacagtggaagtcccatcggagctatagctgccaagtcacccacgagggctccaccgtggaaaagactgtcgcccccaccgaatgttcg

SEQ ID NO: 13 - DNA, 1359 bp, synthetic construct
  source 1..1359: mol_type = other DNA
caagtacagttgcagcagtccggagcggaagtcaagaagccgggctccagcgtgcgcgtgtcctgtaaagccagcggcggcaccttcaacaacgatgccattaactgggtccgccaggccccgggacagggtcttgaatggatgggcgggatcttcccgcttttcggaactgcaaagtacagccaaaacttccagggccgggtggccatcacagctgacgagtcaaccggaaccgcctccatggagctctcttcgctccgctcggaagatactgccgtgtattactgcgcccggtcacgggacttgctcctgttcccacaccatgctctgtccccctggggcagagggaccatggtcactgtgtcctcggcatccactaaggggcctagcgtctttccgctggccccgtcctccaagtccacttcgggtggaaccgcggcactggggtgcctcgtgaaggactacttccccgagccggtcaccgtgtcctggaactcgggagccctgacctccggagtgcatactttccctgcggtgctgcagtcctccgggctctactcgctgtcaagcgtggtcaccgtcccgagctcatccctgggtactcagacctacatttgcaacgtgaaccacaaaccttccaacaccaaggtcgacaagaaagtggagcctaagagctgcgacaagacccacacctgtcccccgtgtcccgcccctgagctgctgggcggccccagcgtgttcctcttcccgcctaagccgaaggacactctgtacatcaccagagagcctgaagtgacctgtgtggtggtggatgtgtcccacgaggatccggaagtgaagttcaattggtacgtggacggagtggaagtccataacgccaagaccaagccccgcgaggaacagtacaactcaacttaccgggtggtgtcagtgctgaccgtgctgcaccaagattggctgaacgggaaggagtacaagtgcaaagtctccaacaaggcgctgccggcccccattgaaaagaccatcagcaaggctaagggccagccccgggaaccacaggtctacaccttgcccccttcccgggacgaactgaccaagaaccaagtgtcgctgacgtgcctggtcaagggcttttatccatctgacatcgccgtggagtgggaaagcaacggccagccggaaaacaactacaagactaccccgcctgtgctggactccgacggctcgttcttcctgtattccaagctcaccgtggataagtccagatggcagcagggcaatgtgttcagctgcagcgtgatgcatgaggccctgcacaaccactacactcagaaatcactgtccctttcccccggaaaa

SEQ ID NO: 14 - DNA, 642 bp, synthetic construct
  source 1..642: mol_type = other DNA
tcgtccgaactgacccaggacccggctgtgtccgtggccctgggacaaactgtgcggatcacgtgccagggcgacttcctgcgctccaactacgcgtcgtggtaccagcaaaagcctgggcaggcgccggtgttggtgatctacggaaagaacaatcgcccctccggaattccagacaggttcagcggcagctcatccgggaacaccgcctcactgaccatcaccggagcccaggccgaagatgaagctgactactactgctcctcgagagattccagcggcaaccactgggtgtttggtggcggaacagagctcactgtgctcgggcagccgaaggctgcgccctcggtgacccttttcccgccatcatccgaggagctgcaggccaacaaggccacgctcgtgtgcctgatctcggatttctaccctggtgccgtgactgtggcctggaaggcagactctagccccgtgaaggccggagtggagactaccaccccgtccaagcagtccaacaacaaatacgccgcgtcatcctacctgtccctgacccctgaacagtggaagtcccatcggagctatagctgccaagtcacccacgagggctccaccgtggaaaagactgtcgcccccaccgaatgttcg

SEQ ID NO: 15 - protein, 5 aa, synthetic construct
NDAIN

SEQ ID NO: 16 - protein, 17 aa, synthetic construct
GIFPLFGTAKYSQNFQG

SEQ ID NO: 17 - protein, 14 aa, synthetic construct
SRDLLLFPHHALSP

SEQ ID NO: 18 - protein, 11 aa, synthetic construct
QGDFLRSNYAS

SEQ ID NO: 19 - protein, 7 aa, synthetic construct
GKNNRPS

SEQ ID NO: 20 - protein, 11 aa, synthetic construct
SSRDSSGNHWV

SEQ ID NO: 21 - protein, 123 aa, synthetic construct
QVQLQQSGAEVKKPGSSVRVSCKASGGTFNNDAINWVRQAPGQGLEWMGGIFPLFGTAKYSQNFQGRVAITADESTGTASMELSSLRSEDTAVYYCARSRDLLLFPHHALSPWGRGTMVTVSS

SEQ ID NO: 22 - protein, 108 aa, synthetic construct
SSELTQDPAVSVALGQTVRITCQGDFLRSNYASWYQQKPGQAPVLVIYGKNNRPSGIPDRFSGSSSGNTASLTITGAQAEDEADYYCSSRDSSGNHWVFGGGTELTVL

SEQ ID NO: 23 - protein, 453 aa, synthetic construct
QVQLQQSGAEVKKPGSSVRVSCKASGGTFNNDAINWVRQAPGQGLEWMGGIFPLFGTAKYSQNFQGRVAITADESTGTASMELSSLRSEDTAVYYCARSRDLLLFPHHALSPWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, synthetic construct
SSELTQDPAVSVALGQTVRITCQGDFLRSNYASWYQQKPGQAPVLVIYGKNNRPSGIPDRFSGSSSGNTASLTITGAQAEDEADYYCSSRDSSGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2026/0285996 A1
(43) Pub. Date: Sep. 24, 2026

(54) BAFF SPECIFIC ANTIBODIES

(71) Applicant: GlaxoSmithKline Intellectual Property Development Limited, Stevenage (GB)

(72) Inventors: Farheen AHMED, Stevenage (GB); Martin ORECCHIA, Stevenage (GB); Milan OVECKA, Stevenage (GB)

(73) Assignee: GlaxoSmithKline Intellectual Property Development Limited, Stevenage (GB)

(21) Appl. No.: 19/681,490

(22) Filed: May 19, 2026

Related U.S. Application Data
(63) Continuation of application No. PCT/EP2024/085230, filed on Dec. 9, 2025.
(60) Provisional application No. 63/608,374, filed on Dec. 11, 2023.

(51) Int. Cl. C07K 16/28; A61K 39/00

(52) U.S. Cl. CPC C07K 16/2875; A61K 2039/505; C07K 2317/33; C07K 2317/52; C07K 2317/55; C07K 2317/565; C07K 2317/622; C07K 2317/92

(57) ABSTRACT

The present invention relates to anti-BLyS antigen binding proteins such as an anti-BLyS antibody which is capable of antagonizing BLyS. The invention also relates to nucleic acid sequences which encode the antigen binding protein as well as the use the antigen binding protein in therapy, for example, in diseases in which B cells play a pathogenic role, such as, for example, in autoimmune diseases, chronic inflammatory diseases and/or cancer.

### CROSS-REFERENCE TO RELATED APPLICATIONS

This application is a continuation of International Application No. PCT/EP2024/085230, filed Dec. 9, 2024, which claims priority to and benefit of U.S. Provisional Application No. 63/608,374, filed Dec. 11, 2023, both of which are incorporated by reference herein in their entireties.

### SEQUENCE LISTING SUBMITTED ELECTRONICALLY

This application contains a sequence listing, which is provided in XML format with a file name “70298WO01.xml”. The XML file has a size of 28,672 bytes and was created on Oct. 16, 2024. The sequence listing submitted electronically is part of the specification and is incorporated herein by reference in its entirety.

### FIELD OF THE INVENTION

The present invention relates to anti-BLyS antigen binding proteins, such as monoclonal antibodies. The invention also includes pharmaceutical compositions comprising them and methods of treatment and uses of such binding proteins.

### BACKGROUND TO THE INVENTION

BLyS (also known as BAFF) is a member of the TNF ligand superfamily (Schneider P et al., J Exp Med. 1999 Jun. 7; 189(11):1747-56) and well characterized as an essential survival factor for peripheral B cells and promotes naïve B cell differentiation to immunoglobulin-producing plasma cells. BLyS is a homotrimeric type II transmembrane protein and exists as both membrane and extracellular secreted forms. In human monocytes, membrane BLyS is detected on the cell surface, and is constitutively cleaved by furin protease to be released from the cells. Soluble BLyS trimers are the predominant form detected in human serum, although these may further oligomerize in vitro through trimer-trimer interactions to form higher order 60-mer structures. Whether BLyS 60-mer is significantly present in vivo however is under active investigation.

BLyS binds to three receptors (BAFFR (BR3), TACI, and BCMA) which are expressed at different stages of B cell development. BAFFR is highly expressed in transitional and naïve B cells and mediates expression of anti-apoptotic genes enabling B cell survival. Consistent with this, inhibition or loss of BLyS leads to reduced circulating naïve B cells. In contrast, TACI and BCMA are expressed during B cell activation and differentiation to plasma cells. TACI signalling stimulates class switch recombination and is implicated in generation of autoreactive B cells in diseased tissue via the T cell independent, so-called ‘extrafollicular’ pathway. Whilst BLyS is the only ligand for BAFFR, a related ligand (APRIL) also binds TACI and BCMA; thus, there may be redundancy for BLyS signalling via TACI and BCMA, and the specific disease and tissue context are likely to impact the relative roles of BLyS and APRIL on these receptors. BCMA is enriched on plasma cells and with APRIL probably plays a role in maintenance of long-lived plasma cells in bone marrow.

BLyS is predominantly expressed by myeloid cells but may also be produced by fibroblasts and other immune cells and is induced by inflammatory cytokines, such as interferons and GM-CSF. Dysregulated increases in BLyS are associated with a break in peripheral B cell tolerance and the development of autoimmunity in vivo. Mice lacking BLyS have impaired B cell maturation and lack mature splenic B cells. Conversely, mice which constitutively over express BLyS (BLyS transgenic mice) have increased numbers of peripheral B cells, increased production of autoantibodies, proteinuria, and glomerulonephritis, suggesting that it is potentially both necessary and sufficient for disease initiation and progression. The impact of BLyS blockade in autoimmune disease models has also been explored using soluble BLyS receptors (TACI-Fc or BAFFR-Fc) in New Zealand Black/New Zealand White (NZB/NZW) F1 mice (which develop a lethal, systemic lupus erythematosus (SLE)-like autoimmune syndrome), where treatment slowed disease progression, and improved survival.

In humans, BLyS is over expressed in the serum of patients with SLE and other autoimmune diseases (reviewed in Cancro M P et al. J Clin Invest. 2009 May; 119(5):1066-73). Furthermore, BLyS levels in rheumatoid arthritis (RA) and SLE patients have been found to positively correlate with autoantibody levels, immunoglobulin IgG, and disease activity.

Systemic lupus erythematosus (SLE) is a chronic autoimmune disease with clinical and serological heterogeneity and is associated with production of autoantibodies which eventually leads to chronic inflammation and irreversible organ damage. Lupus Nephritis (LN) is one of the more severe manifestations of SLE and approximately 40% of SLE patients will develop LN over the course of their disease, with 80-90% progressing to LN within 5 years of SLE diagnosis. Of those patients with SLE, approximately 30-50% are deemed to be sub-optimally treated and will develop irreversible organ damage within 5 years of diagnosis due to uncontrolled disease. Approximately 22% of these patients in developed countries progress to end stage kidney disease by year 15. The marketed antibody belimumab, a recombinant, fully human, IgG1λ monoclonal antibody which selectively binds and neutralizes soluble BLyS trimer has been approved for the treatment of SLE and LN and is in clinical trials for further indications.

Despite the approval of belimumab, new treatments for systemic lupus erythematosus (SLE) and lupus nephritis (LN) as well as other diseases where B cells play a pathogenic role, are needed to reduce irreversible organ damage and steroid use (~54% of SLE patients are prescribed steroids).

The risk of mortality in patients with LN is approximately 6-fold higher than the general population, due to the risk of renal failure and treatment-related complications. Despite the current availability of beneficial treatment options for patients with SLE and LN, there is a need for additional treatment options in these patient populations. Specifically, numerous retrospective analyses have established that real-world medication adherence in SLE is low, with estimated 1-year adherence and persistence approximately 47% and 30% to 36%, respectively. Medication non-adherence in SLE has been associated with increased all-cause and SLE-related emergency department visits and hospitalizations in addition to increased health-care costs. It is hypothesized that increasing the dosing interval for medications in SLE could improve persistence to treatment and support longer-term patient outcomes. Additionally, new treatments for SLE and LN are needed to reduce irreversible organ damage and to reduce the need for steroid use.

There is therefore a need not only for improved treatments for such diseases but also for treatment options with reduced injection frequency compared to other approved biologics and those currently in development to aid patient compliance and to lead to improved patient outcomes.

### SUMMARY OF THE INVENTION

In one aspect, there is provided a BLyS binding protein comprising any one or a combination of CDRs selected from CDRH1 of SEQ ID NO:1, CDRH2 of SEQ ID NO:2, CDRH3 of SEQ ID NO:3, CDRL1 of SEQ ID NO: 4, CDRL2 of SEQ ID NO:5, and/or CDRL3 of SEQ ID NO:6.

In one aspect, there is provided a BLyS binding protein comprising the following 6 CDRs: CDRH1 of SEQ ID NO: 1, CDRH2 of SEQ ID NO:2, CDRH3 of SEQ ID NO:3, CDRL1 of SEQ ID NO:4, CDRL2 of SEQ ID NO: 5, and CDRL3 of SEQ ID NO:6.

In a second aspect, there is provided a BLyS binding protein comprising any one or a combination of CDRs selected from CDRH1 of SEQ ID NO:15, CDRH2 of SEQ ID NO: 16, CDRH3 of SEQ ID NO:17, CDRL1 of SEQ ID NO:18, CDRL2 of SEQ ID NO:19, and/or CDRL3 of SEQ ID NO:20.

In one aspect, there is provided a BLyS binding protein comprising the following 6 CDRs: CDRH1 of SEQ ID NO: 15, CDRH2 of SEQ ID NO:16, CDRH3 of SEQ ID NO: 17, CDRL1 of SEQ ID NO:18, CDRL2 of SEQ ID NO: 19, and CDRL3 of SEQ ID NO:20.

In a third aspect, there is provided nucleic acid sequence(s) which encode one or both of the heavy and/or light chains of the BLyS binding protein as defined herein.

In one aspect, there is provided a nucleic acid sequence which encodes a BLyS binding protein as defined herein.

In a fourth aspect, there is provided expression vector(s) comprising one or more nucleic acid sequence(s) as defined herein.

In a fifth aspect, there is provided recombinant host cell(s) comprising nucleic acid sequence(s) or expression vector(s) as defined herein.

In a sixth aspect, there is provided a method for the production of one or more BLyS binding proteins, which method comprises culturing the recombinant host cell(s) as defined herein under conditions suitable for expression of said nucleic acid sequence(s) or expression vector(s), whereby a polypeptide comprising the BLyS binding protein is produced.

In a seventh aspect, there is provided pharmaceutical composition(s) comprising the BLyS binding protein as defined herein and a pharmaceutically acceptable excipient.

In an eighth aspect, there is provided methods for the treatment of diseases where B cells play a pathogenic role in a human in need thereof comprising administering to said human a therapeutically effective amount of the BLyS binding protein(s) or the pharmaceutical composition(s) comprising the BLyS binding protein(s) to the subject.

In a ninth aspect, there is provided the BLyS binding protein(s) as defined herein or the pharmaceutical composition(s) comprising the BLyS binding protein for use in therapy.

In a tenth aspect, there is provided the BLyS binding protein(s) or pharmaceutical composition(s) comprising the BLyS binding protein(s) for use in the manufacture of a medicament for use in the treatment of diseases where B cells play a pathogenic role.

### DESCRIPTION OF DRAWINGS/FIGURES

FIG. 1—Octet sensorgram showing effect of mutating key residues on the binding of belimumab. OctetRED Protein A Sensors were hydrated in perfluorobutanesulfonyl fluoride (PBSF) for 10 minutes prior to use. Belimumab (20 μg/mL) was captured on sensor tips (60 s). Followed by a wash (10 s) and baseline step (60 s). Epofix (huIgG1, 50 μg/mL) was used to block the sensors (blocking step, 240 s) followed by second wash (10 s) and baseline steps (60 s). Association (individual human BAFF (hBAFF) proteins, 64 nM nominal concentration based on info supplied with samples) was 240 seconds followed by dissociation into PBSF for 300 seconds. Sensors were regenerated using 10 mM glycine pH 1.5 (Sodexo) throughout the run. Plate temperature was 25° C., 1000 rpm plate shaker speed. Data was analysed using ForteBio Data Analysis V.4.

FIG. 2—Screening of the hit list clones for binding to recombinant human BAFF at 37° C. was performed using capture kinetics method on Biacore T200 (Cytiva) surface plasmon resonance instrument. The antibodies, including controls, were captured on anti-human Fc capture antibody (MP Biomedicals) which was immobilised on flow cells 2 of a CM5 series S sensor chip by primary amine coupling. The BAFF analytes were injected at injection rate of 30 μL/min over the captured antibodies as a concentration series including two 2× serial dilutions. The concentrations of analyte were 16 nM and 4 nM. Association phase was 240 seconds, followed by a dissociation phase of 600 seconds. Buffer only (0 nM analyte) injection was used to double reference the binding curves. Regeneration of the chip surface between cycles was carried out using 10 mM Glycine pH 1.5. The assay was run at 37° C. in HBS-EP⁺ buffer.

FIG. 3—shows the crystal structure of a BLyS trimer (displayed in black ribbon representation) bound to three Ab363A Fab fragments (displayed in grey (heavy chain) and light grey (light chain) surface representation) at 2.23 Å resolution. The Fabs are bound at equivalent epitopes around the BLyS trimer. An average interface area of 1,025 Å² and buried surface area of 2319 Å² for each Fab with the BLyS trimer were determined using PISA and PyMOL respectively. Considering the amount of interface surface area available to confer binding, the affinity of Ab363A is exceptionally high and exceeds the binding efficiencies of all comparable protein-protein interactions in two benchmark studies.

FIG. 4—shows the BLyS trimer epitope (displayed in surface representation) that interacts with an individual Ab363A Fab, within a contact radius of 4.5 Å using PYMOL. The epitope is formed from two BLyS protomers of the trimer: a principal protomer providing most of the epitope (shown in black) and comprising K¹⁶⁰G¹⁶¹S¹⁶²Y¹⁶³T²⁰⁵Y²⁰⁶A²⁰⁷M²⁰⁸G²⁰⁹K²¹⁵G²²¹D²²²L²²⁴S²²⁵L²²⁶R²³¹I²³³P²⁶⁴R²⁶⁵ E²⁶⁶, while an adjacent BLyS protomer of the trimer extends the epitope with L²⁴⁰N²⁴² (shown in light grey).

FIG. 5—shows the Ab363A Fab paratope (displayed in surface representation) interacting with the BLyS trimer within a contact radius of 4.5 Å using PYMOL. The Fab heavy chain contributes N³¹M⁵⁴F⁵⁵G⁵⁶T⁵⁷K⁵⁹D¹⁰¹P¹⁰²L¹⁰³L¹⁰⁴ (shown in black) and the light chain L²⁷R²⁸Y²⁹Y³⁰Y³¹K⁵⁰S⁶⁴S⁶⁵S⁶⁶S⁹³G⁹⁴N⁹⁵ (shown in light grey).

FIG. 6—shows surface representation (using PYMOL) of CDR_H2 F52 highlighting its space filling and pi-stacking properties and proximity to CDRH3 P102 in the Ab363A Fab paratope.

FIG. 7A—shows CDRL1 Y29 sidechain space filling the pocket where it resides and additionally hydrogen-bonding to the CDRL1 R28 in the Ab363A Fab paratope (displayed in surface representation) using PYMOL.

FIG. 7B—provides labeling of the residues shown in FIG. 7A.

FIG. 8—shows an overlay of the three Ab363A Fab/BLyS protomer subunits of the Ab363A Fab/BLyS trimer structure. The subunits are coloured black, grey, and light grey and the overlay highlights the close structural similarity of the three Ab363A Fab/BLyS contact interfaces in the trimer. BLyS is shown with a representative molecular surface from one of the overlaid BLyS protomers.

FIG. 9—shows an overlay of Ab363A Fab/BLyS (black) and belimumab Fab/BLyS (light grey) crystal structures. It highlights the comparable 3-fold symmetry, interaction geometry and stoichiometry. The belimumab Fab/BLyS complex is obtained from PDB entry 5y9j.

FIG. 10—shows a comparison of BLyS epitopes between Ab363A and belimumab. Epitope identified using a 4.5 Å interaction cut-off. BlyS residues from the three distinct regions (receptor binding site on BlyS, BlyS flap region and contact with adjacent BlyS protomer of trimer) of the epitope, are indicated [shaded in grey]. Epitope for Belimumab was obtained from PDB entry 5y9j.

FIG. 11—shows serum concentrations of Ab363A following intravenous administration at 0.5 mg/kg in the cynomolgus monkey.

FIG. 12—shows serum concentrations of Ab363A following intravenous administration at 2.0 mg/kg in the cynomolgus monkey.

### DETAILED DESCRIPTION OF THE INVENTION

In one aspect, there is provided a BLyS binding protein. The term “BLyS binding protein” as used herein refers to antibodies, antigen binding fragments thereof, and other protein constructs, such as domains, which are capable of binding to BLyS. A BLyS binding protein can be capable of binding to a fragment of, a variant of, or a mutant of BLyS.

In one aspect, there is provided a BLyS binding protein comprising any one or a combination of CDRs selected from CDRH1 of SEQ ID NO:1, CDRH2 of SEQ ID NO:2, CDRH3 of SEQ ID NO:3, CDRL1 of SEQ ID NO: 4, CDRL2 of SEQ ID NO:5, and/or CDRL3 of SEQ ID NO:6.

In one aspect, there is provided a BLyS binding protein comprising CDRH1 of SEQ ID NO:1, CDRH2 of SEQ ID NO:2, and CDRH3 of SEQ ID NO:3. In one aspect, there is provided a BLyS binding protein comprising CDRL1 of SEQ ID NO:4, CDRL2 of SEQ ID NO:5, and CDRL3 of SEQ ID NO:6.

In a further aspect, there is provided a BLyS binding protein comprising CDRH1 of SEQ ID NO:1, CDRH2 of SEQ ID NO:2, CDRH3 of SEQ ID NO:3, CDRL1 of SEQ ID NO:4, CDRL2 of SEQ ID NO:5, and CDRL3 of SEQ ID NO:6.

In a further aspect, there is provided a BLyS binding protein comprising a variable heavy (VH) region at least 80% identical to the sequence of SEQ ID NO:7 and/or a variable light (VL) region at least 80% identical to the sequence of SEQ ID NO:8, wherein CDRH1 comprises SEQ ID NO:1, CDRH2 comprises SEQ ID NO:2, CDRH3 comprises SEQ ID NO:3, CDRL1 comprises SEQ ID NO:4, CDRL2 comprises SEQ ID NO: 5, and CDRL3 comprises SEQ ID NO:6. In a further aspect, there is provided a BLyS binding protein comprising a VH region at least 90% identical to the sequence of SEQ ID NO:7 and/or a VL region at least 90% identical to the sequence of SEQ ID NO:8, wherein CDRH1 comprises SEQ ID NO:1, CDRH2 comprises SEQ ID NO:2, CDRH3 comprises SEQ ID NO:3, CDRL1 comprises SEQ ID NO:4, CDRL2 comprises SEQ ID NO:5, and CDRL3 comprises SEQ ID NO:6. In a further aspect, there is provided a BLyS binding protein comprising a VH region at least 95% identical to the sequence of SEQ ID NO:7 and/or a VL region at least 95% identical to the sequence of SEQ ID NO:8, wherein CDRH1 comprises SEQ ID NO:1, CDRH2 comprises SEQ ID NO:2, CDRH3 comprises SEQ ID NO:3, CDRL1 comprises SEQ ID NO: 4, CDRL2 comprises SEQ ID NO:5, and CDRL3 comprises SEQ ID NO:6. In a further aspect, there is provided a BLyS binding protein comprising a VH region at least 98% identical to the sequence of SEQ ID NO: 7 and/or a VL region at least 98% identical to the sequence of SEQ ID NO:8, wherein CDRH1 comprises SEQ ID NO:1, CDRH2 comprises SEQ ID NO:2, CDRH3 comprises SEQ ID NO:3, CDRL1 comprises SEQ ID NO:4, CDRL2 comprises SEQ ID NO:5, and CDRL3 comprises SEQ ID NO:6. In a further aspect, there is provided a BLyS binding protein comprising a VH region at least 99% identical to the sequence of SEQ ID NO:7 and/or a VL region at least 99% identical to the sequence of SEQ ID NO:8, wherein CDRH1 comprises SEQ ID NO:1, CDRH2 comprises SEQ ID NO:2, CDRH3 comprises SEQ ID NO: 3, CDRL1 comprises SEQ ID NO:4, CDRL2 comprises SEQ ID NO:5, and CDRL3 comprises SEQ ID NO: 6.

In one aspect, there is provided a BLyS binding protein comprising a VH region at least 80%, at least 85%, at least 90%, at least 95%, at least 96%, at least 97%, at least 98%, or at least 99% identical to the sequence of SEQ ID NO:7, wherein CDRH1 comprises SEQ ID NO:1, CDRH2 comprises SEQ ID NO:2, and CDRH3 comprises SEQ ID NO:3; and/or a VL region at least 80%, at least 85%, at least 90%, at least 95%, at least 96%, at least 97%, at least 98%, or at least 99% identical to the sequence of SEQ ID NO: 8, wherein CDRL1 comprises SEQ ID NO:4, CDRL2 comprises SEQ ID NO:5, and CDRL3 comprises SEQ ID NO:6. In one aspect, the BLyS binding protein comprises a VH region at least 80%, at least 85%, at least 90%, at least 95%, at least 96%, at least 97%, at least 98%, or at least 99% identical to the sequence of SEQ ID NO:7, wherein CDRH1 comprises SEQ ID NO:1, CDRH2 comprises SEQ ID NO:2, and CDRH3 comprises SEQ ID NO:3; and/or a VL region at least 80%, at least 85%, at least 90%, at least 95%, at least 96%, at least 97%, at least 98%, or at least 99% identical to the sequence of SEQ ID NO: 8, wherein CDRL1 comprises SEQ ID NO:4, CDRL2 comprises SEQ ID NO:5, and CDRL3 comprises SEQ ID NO:6, and wherein the VL region further comprises a serine residue at position 64 and/or 65 and/or 66 of SEQ ID NO:8. In one aspect, the VL region comprises a serine residue at each of positions 64, 65 and 66 of SEQ ID NO:8.

For the avoidance of doubt, the variability in the sequence homology/identity does not occur in the CDRS.

In one such aspect, there is provided a BLyS binding protein comprising a VH region of SEQ ID NO:7 and/or a VL region of SEQ ID NO:8. In one such aspect, there is provided a BLyS binding protein comprising a VH region of SEQ ID NO:7 and a VL region of SEQ ID NO:8.

In one aspect, there is provided a BLyS binding protein comprising any one or a combination of CDRs selected from CDRH1 of SEQ ID NO:15, CDRH2 of SEQ ID NO:16, CDRH3 of SEQ ID NO:17, CDRL1 of SEQ ID NO:18, CDRL2 of SEQ ID NO: 19, and/or CDRL3 of SEQ ID NO:20.

In a further aspect, there is provided a BLyS binding protein comprising CDRH1 of SEQ ID NO:15, CDRH2 of SEQ ID NO:16, CDRH3 of SEQ ID NO:17, CDRL1 of SEQ ID NO:18, CDRL2 of SEQ ID NO:19, and CDRL3 of SEQ ID NO:20.

In a further aspect, there is provided a BLyS binding protein comprising a VH region at least 80% identical to the sequence of SEQ ID NO:21 and/or a VL region at least 80% identical to the sequence of SEQ ID NO:22, wherein CDRH1 comprises SEQ ID NO:15, CDRH2 comprises SEQ ID NO:16, CDRH3 comprises SEQ ID NO: 17, CDRL1 comprises SEQ ID NO:18, CDRL2 comprises SEQ ID NO:19, and CDRL3 comprises SEQ ID NO:20. In a further aspect, there is provided a BLyS binding protein comprising a VH region at least 90% identical to the sequence of SEQ ID NO:21 and/or a VL region at least 90% identical to the sequence of SEQ ID NO:22, wherein CDRH1 comprises SEQ ID NO:15, CDRH2 comprises SEQ ID NO: 16, CDRH3 comprises SEQ ID NO: 17, CDRL1 comprises SEQ ID NO:18, CDRL2 comprises SEQ ID NO: 19, and CDRL3 comprises SEQ ID NO:20. In a further aspect, there is provided a BLyS binding protein comprising a VH region at least 95% identical to the sequence of SEQ ID NO:21 and/or a VL region at least 95% identical to the sequence of SEQ ID NO:22, wherein CDRH1 comprises SEQ ID NO: 15, CDRH2 comprises SEQ ID NO:16, CDRH3 comprises SEQ ID NO: 17 and CDRL1 comprises SEQ ID NO: 18, CDRL2 comprises SEQ ID NO: 19, and CDRL3 comprises SEQ ID NO:20. In a further aspect, there is provided a BLyS binding protein comprising a VH region at least 98% identical to the sequence of SEQ ID NO: 21 and/or a VL region at least 98% identical to the sequence of SEQ ID NO:22, wherein CDRH1 comprises SEQ ID NO:15, CDRH2 comprises SEQ ID NO:16, CDRH3 comprises SEQ ID NO:17, CDRL1 comprises SEQ ID NO: 18, CDRL2 comprises SEQ ID NO: 19, and CDRL3 comprises SEQ ID NO:20. In a further aspect, there is provided a BLyS binding protein comprising a VH region at least 99% identical to the sequence of SEQ ID NO: 21 and/or a VL region at least 99% identical to the sequence of SEQ ID NO: 22, wherein CDRH1 comprises SEQ ID NO:15, CDRH2 comprises SEQ ID NO: 16, CDRH3 comprises SEQ ID NO:17, CDRL1 comprises SEQ ID NO:18, CDRL2 comprises SEQ ID NO: 19, and CDRL3 comprises SEQ ID NO:20.

For the avoidance of doubt, the variability in sequence identity does not occur in the CDRs.

In one such aspect, there is provided a BLyS binding protein comprising a VH region of SEQ ID NO:21 and/or a VL region of SEQ ID NO:22. In one such aspect, there is provided a BLyS binding protein comprising a VH region of SEQ ID NO:21 and a VL region of SEQ ID NO:22.

In another aspect, the BLyS binding protein as herein described contains an Fc region. In a further aspect, the BLyS binding protein as herein described comprises a modified Fc region. In a further aspect, the Fc region or modification in the Fc region confers increased half-life on the BLyS binding protein as disclosed herein. As used herein, the term increased half-life refers to an increase in the time required for the serum concentration of an antigen binding protein to reach half of its original value relative to a wild type antigen binding protein, in particular, an IgG1 antibody that does not contain modifications to its Fc region when measured in an FcRn binding assay.

A number of mechanisms are described throughout to increase half-life and are considered aspects of the invention as herein described.

In one aspect, the BLyS binding protein as herein described comprises a heavy chain Fc domain having a tyrosine residue at position 252, a threonine residue at position 254, and a glutamic acid residue at position 256 (EU index numbering). In another aspect, the BLyS binding protein as herein described comprises a heavy chain Fc domain having a leucine residue as position 428 and a serine residue at position 434 (EU index numbering).

In an alternative aspect, the BLyS binding protein as herein described comprises a heavy chain Fc domain having a lysine residue as position 433 and a phenylalanine residue at position 434 (EU index numbering).

In one aspect, there is provided a BLyS binding protein comprising a heavy chain at least 80%, at least 85%, at least 90%, at least 95%, at least 96%, at least 97%, at least 98%, or at least 99% identical to the sequence of SEQ ID NO:9 and/or a light chain at least 80%, at least 85%, at least 90%, at least 95%, at least 96%, at least 97%, at least 98%, or at least 99% identical to the sequence of SEQ ID NO: 10, wherein CDRH1 comprises SEQ ID NO:1, CDRH2 comprises SEQ ID NO:2, CDRH3 comprises SEQ ID NO: 3, CDRL1 comprises SEQ ID NO:4, CDRL2 comprises SEQ ID NO:5, and CDRL3 comprises SEQ ID NO: 6.

In a further aspect, there is provided a BLyS binding protein comprising a heavy chain at least 80%, at least 85%, at least 90%, at least 95%, at least 96%, at least 97%, at least 98%, or at least 99% identical to the sequence of SEQ ID NO:23 and/or a light chain at least 80%, at least 85%, at least 90%, at least 95%, at least 96%, at least 97%, at least 98%, or at least 99% identical to the sequence of SEQ ID NO: 24, wherein CDRH1 comprises SEQ ID NO:15, CDRH2 comprises SEQ ID NO:16, CDRH3 comprises SEQ ID NO:17, CDRL1 comprises SEQ ID NO:18, CDRL2 comprises SEQ ID NO: 19, and CDRL3 comprises SEQ ID NO:20.

For the avoidance of doubt, the variability in sequence identity does not occur in the CDRs.

In one aspect, there is provided a BLyS binding protein as herein described wherein the BLyS binding protein comprises a heavy chain of SEQ ID NO:9 and/or a light chain of SEQ ID NO:10. In one aspect, there is provided a BLyS binding protein as herein described wherein the BLyS binding protein comprises a heavy chain of SEQ ID NO:9 and a light chain of SEQ ID NO: 10. In one aspect, there is provided a BLyS binding protein as herein described wherein the BLyS binding protein comprises a heavy chain of SEQ ID NO: 23 and/or a light chain of SEQ ID NO:24. In one aspect, there is provided a BLyS binding protein as herein described wherein the BLyS binding protein comprises a heavy chain of SEQ ID NO:23 and a light chain of SEQ ID NO:24.

In one aspect, there is provided a BLyS binding protein comprising: a VH region at least 80%, at least 85%, at least 90%, at least 95%, at least 96%, at least 97%, at least 98%, or at least 99% identical to the sequence of SEQ ID NO:7; and a VL region at least 80%, at least 85%, at least 90%, at least 95%, at least 96%, at least 97%, at least 98%, or at least 99% identical to the sequence of SEQ ID NO:8, wherein the BLyS binding protein comprises a CDRH1, CDRH2 and CDRH3 selected from (i) or (ii) below; and a CDRL1, CDRL2 and CDRL3 selected from (iii) or (iv) below:
(i) a CDRH1, CDRH2 and/or CDRH3 from SEQ ID NO:7, or (ii) a CDR variant of (i) having 1, 2 or 3 amino acid modifications in one or more of CDRH1, CDRH2 and/or CDRH3, and wherein the BLyS binding protein comprises N31, M54, F55, G56, T57, K59, D101, P102, L103 and L104 of SEQ ID NO:7; and
(iii) a CDRL1, CDRL2 and/or CDRL3 from SEQ ID NO:8, or (iv) a CDR variant of (iii) having 1, 2 or 3 amino acid modifications in one or more of CDRL1, CDRL2 and/or CDRL3, and wherein the BLyS binding protein comprises L27, R28, Y29, Y30, Y31, K50, S93, G94, and N95 of SEQ ID NO:8.

In one embodiment, the aforementioned CDRs are numbered according to the Kabat numbering convention. In one embodiment, the VL region further comprises S64, S65 and/or S66 of SEQ ID NO:8. In one embodiment, the VL region further comprises S64, S65 and S66 of SEQ ID NO:8.

In one aspect, the BLyS binding protein is an antibody. In a further aspect, the BLyS binding protein is a monoclonal antibody. In a further aspect, the BLyS binding protein is an IgG1 antibody. In yet a further aspect, the BLyS binding protein is an IgG1 monoclonal antibody.

In one aspect, the BLyS binding protein as disclosed herein is a bispecific antibody. In a further aspect, the bispecific antibody is a Duobody, L-body, Common light chain antibody, antibody with modified cysteine bridging between the heavy and light chains, Chimeric heavy/light chain antibody, Cross-Mab, mAb pair, or Het-mAb. In a further aspect, the BLyS binding protein is a Het-mAb.

As used herein, a “Het-mAb” is an IgG-like molecule that can target two different epitopes, either on the same or different targets, with 4 distinct chains: 2 heavy and 2 light. These chains contain a set of mutations in the Fc portion of the molecule to drive heavy chain dimerization and a set of mutations on the FAb portion to drive correct heavy/light pairing that form kappa/kappa or lambda/kappa subtype bispecific mAbs. Suitable mutations for driving heavy chain dimerization are disclosed in WO2012/058768 and WO2013/063702. Suitable mutations for driving heavy chain/light chain (HC/LC) pairing are disclosed in WO 2014/082179, WO 2015/181805, and WO 2017/059551. In one aspect, the heavy chains of both the BLyS binding protein and the other binding protein contain mutations which direct the correct pairing of the heavy chains. In one aspect, the FAb portions of the heavy and light chains contain mutations which direct the correct pairing of the heavy and light chains.

In one aspect, the BLyS binding protein as disclosed herein binds to BLyS, for example, to human BLyS with an equilibrium dissociation constant (KD) of less than 1000 pM, less than 500 pM, less than 250 pM, less than 150 pM, less than 100 pM, or less than 50 pM (for example, 39 pM). In one embodiment, the BLyS binding protein as disclosed herein binds to BLyS, for example, to human BLyS with an equilibrium dissociation constant (KD) of less than or equal to 150 pM, less than or equal to 140 pM, less than or equal to 130 pM, less than or equal to 120 pM, less than or equal to 110 pM, less than or equal to 100 pM, less than or equal to 90 pM, less than or equal to 80 pM, less than or equal to 70 pM, less than or equal to 60 pM, less than or equal to 50 pM, or less than or equal to 40 pM (for example, 39 pM).

The BLyS binding proteins of the invention are capable of antagonizing BLyS and may decrease or inhibit BLyS-induced signal transduction. For example, BLyS binding proteins are capable of disrupting the interaction between BLyS and its receptor to inhibit or downregulate BLyS-induced signal transduction. In particular, BLyS binding proteins of the invention may prevent BLyS induced signal transduction by specifically recognizing the unbound BLyS protein. The ability of a BLyS binding protein to inhibit or downregulate BLyS induced signal transduction may be determined by techniques known in the art. For example, BLyS-induced receptor activation and the activation of signaling molecules can be determined by detecting the phosphorylation (e.g., tyrosine or serine/threonine) of the receptor or a signaling molecule by immunoprecipitation followed by western blot analysis.

In one aspect, the BLyS binding protein inhibits the binding of human BLyS to its receptor(s). In another aspect, the BLyS binding protein inhibits BLyS binding to BAFFR, BCMA, and TACI. In another aspect, the BLyS binding protein inhibits human BLyS binding to BAFFR, BCMA, and TACI. In a further aspect, the BLyS binding protein inhibits binding of human BLyS trimer to BAFFR, BCMA, and TACI. In one aspect, the BLyS binding protein inhibits binding of human BLyS trimer to BAFFR with an IC50 of 500 pM or less, 250 pM or less, 200 pM or less, 150 pM or less, 100 pM or less, 50 pM or less, 20 pM or less, or 15 pM or less (for example, 13 pM).

In one aspect, the BLyS binding protein inhibits binding of human BLyS trimer to BCMA with an IC50 of 500 pM or less, 250 pM or less, 200 pM or less, 150 pM or less, 100 pM or less, 50 pM or less, 20 pM or less, or 15 pM or less (for example, 13 pM).

In one aspect, the BLyS binding protein inhibits binding of human BLyS trimer to TACI with an IC50 of 500 pM or less, 250 pM or less, 200 pM or less, 150 pM or less, 100 pM or less, 50 pM or less, or 20 pM or less (for example, 16 pM).

In one aspect, the BLyS binding protein inhibits BLyS stimulated CD19+ B cell proliferation. In one aspect, the BLyS binding protein inhibits BLyS stimulated human CD19+ B cell proliferation. In one aspect, the BLyS binding protein inhibits human BLyS trimer-induced human CD19+ primary B cell proliferation. In one such aspect, the BLyS binding protein inhibits BLyS stimulated CD19+ B cell proliferation with an IC50 of 500 pM or less, 250 pM or less, 200 pM or less, 150 pM or less, 100 pM or less, 50 pM or less, 20 pM or less, 15 pM or less, or 10 pM or less (for example, 9.5 pM).

In one aspect, the BLyS binding protein inhibits human BLyS 60mer-stimulated B cell proliferation. In one such aspect, the BLyS binding protein inhibits human BLyS 60mer-stimulated B cell proliferation by IC50 of 1500 pM or less, 1000 pM or less, 500 pM or less, 250 pM or less, 200 pM or less, 150 pM or less, 100 pM or less, or 75 pM or less (for example, 74 pM).

As such, BLyS binding proteins as described herein are potent neutralizers of BLyS-stimulated B cell proliferation. In one aspect, the BLyS binding proteins as described herein are cross reactive with a relevant tox species such as cynomolgus monkey.

In one aspect, the BLyS binding protein inhibits native BLyS stimulated CD19+ B Cell proliferation. In one aspect, the BLyS binding protein inhibits native BLyS stimulated CD19+ B Cell proliferation with an IC50 of 500 pM or less, 250 pM or less, 200 pM or less, 150 pM or less, 100 pM or less, 50 pM or less, 20 pM or less, 10 pM or less, or 5 pM or less (for example, 4.5 pM).

In one aspect, there is provided a nucleic acid sequence which encodes a BLyS binding protein as described herein. In one aspect, there is provided a nucleic acid sequence which encodes one or both of the VH and/or VL region(s) of the BLyS binding protein as described herein. In one aspect, there is provided a nucleic acid sequence which encodes a BLyS binding protein comprising a VH region of SEQ ID NO: 7 and a VL region of SEQ ID NO:8.

In one aspect, there is provided a nucleic acid sequence which encodes one or both of the heavy chain(s) and/or light chain(s) of the BLyS binding protein as described herein. In one aspect, there is provided a nucleic acid sequence which encodes a BLyS binding protein comprising a heavy chain of SEQ ID NO:9 and a light chain of SEQ ID NO:10. In one such aspect, the nucleic acid sequence comprises SEQ ID NO: 11 encoding the heavy chain and/or SEQ ID NO:12 encoding the light chain.

In one aspect, there is provided expression vector(s) comprising nucleic acid sequence(s) which encode the VH and/or VL region(s) of the BLyS binding protein as herein described. In one aspect, there is provided expression vector(s) comprising nucleic acid sequence(s) which encode the heavy and/or light chains of the BLyS binding protein as herein described. In a further aspect, the expression vector(s) express SEQ ID NO: 11 encoding the heavy chain and/or SEQ ID NO:12 encoding the light chain.

In one aspect, there is provided a nucleic acid sequence which encodes one or both of the VH and/or VL region(s) of the BLyS binding protein as described herein. In one aspect, there is provided a nucleic acid sequence which encodes a BLyS binding protein comprising a VH region of SEQ ID NO:21 and a VL region of SEQ ID NO:22. In one aspect, there is provided a nucleic acid sequence which encodes one or both of the heavy chain(s) and/or light chain(s) of the BLyS binding protein as described herein. In one aspect, there is provided a nucleic acid sequence which encodes a BLyS binding protein comprising a heavy chain of SEQ ID NO: 23 and a light chain of SEQ ID NO:24. In one such aspect, the nucleic acid sequence comprises SEQ ID NO: 13 encoding the heavy chain and/or SEQ ID NO:14 encoding the light chain.

In one aspect, there is provided expression vector(s) comprising nucleic acid sequence(s) which encode the VH and/or VL region(s) of the BLyS binding protein as herein described. In one aspect, there is provided expression vector(s) comprising nucleic acid sequence(s) which encode the heavy and/or light chains of the BLyS binding protein as herein described. In a further aspect, the expression vector(s) express SEQ ID NO: 13 encoding the heavy chain and/or SEQ ID NO:14 encoding the light chain.

In another aspect, there is provided a recombinant host cell comprising nucleic acid sequence(s) or expression vector(s) as herein described.

In one aspect, there is provided a method for the production of a BLyS binding protein, which method comprises culturing a host cell as described herein under conditions suitable for expression of said nucleic acid sequence(s) or vector(s), whereby a polypeptide comprising the BLyS binding protein is produced. In a further aspect, there is provided a BLyS binding protein produced by such a method as herein described.

In another aspect, there is provided a cell line engineered to express a BLyS binding protein as disclosed herein.

In one aspect, there is provided a pharmaceutical composition comprising a BLyS binding protein as herein disclosed and a pharmaceutically acceptable excipient.

In one aspect, there is provided a method for the treatment of diseases where B cells play a pathogenic role. In one aspect, the disease may be an autoimmune disease. In another aspect, the disease may be a form of cancer. In one aspect, the disease is a chronic kidney disease or chronic inflammatory disease.

Diseases in which B cells play a pathogenic role may encompass autoimmune diseases such as systemic lupus erythematosus, lupus nephritis, systemic sclerosis, cutaneous lupus erythematosus, anti-neutrophil cytoplasmic antibody (ANCA)-associated vasculitis, systemic sclerosis-associated interstitial lung disease, connective tissue disease, and/or connective tissue disease-associated interstitial lung disease.

In one aspect, there is provided a method for the treatment of an autoimmune disease, a cancer, a chronic kidney disease and/or a chronic inflammatory disease in a human in need thereof comprising administering to said human a therapeutically effective amount of a BLyS binding protein as herein disclosed, or a pharmaceutical composition as disclosed herein comprising the BLyS binding protein. In one aspect, there is provided a method for the treatment of an autoimmune disease and/or a chronic inflammatory disease in a human in need thereof comprising administering to said human a therapeutically effective amount of a BLyS binding protein as herein disclosed, or a pharmaceutical composition as disclosed herein comprising the BLyS binding protein.

In one aspect, there is provided a method for the treatment of systemic lupus erythematosus, lupus nephritis, systemic sclerosis, cutaneous lupus erythematosus, ANCA-associated vasculitis, systemic sclerosis-associated interstitial lung disease, connective tissue disease, and/or connective tissue disease-associated interstitial lung disease in a human in need thereof comprising administering to said human a therapeutically effective amount of a BLyS binding protein as herein disclosed, or a pharmaceutical composition as disclosed herein comprising the BLyS binding protein.

In a further aspect, there is provided a method for the treatment of diseases in which B cells play a pathogenic role.

In a further aspect, there is provided a method for the treatment of a disease where B cells play a pathogenic role. In one such aspect, there is provided a method for the treatment an autoimmune disease, a cancer, a chronic kidney disease and/or a chronic inflammatory disease in a human in need thereof comprising administering to said human a therapeutically effective amount of a BLyS binding protein comprising SEQ ID NO:9 and SEQ ID NO: 10. In one such aspect, there is provided a method for the treatment an autoimmune disease and/or a chronic inflammatory disease in a human in need thereof comprising administering to said human a therapeutically effective amount of a BLyS binding protein comprising SEQ ID NO:9 and SEQ ID NO:10. In one such aspect, there is provided a method for the treatment of systemic lupus erythematosus, lupus nephritis, systemic sclerosis, cutaneous lupus erythematosus, ANCA-associated vasculitis, systemic sclerosis-associated interstitial lung disease, connective tissue disease, and/or connective tissue disease-associated interstitial lung disease in a human in need thereof comprising administering to said human a therapeutically effective amount of a BLyS binding protein comprising SEQ ID NO:9 and SEQ ID NO: 10. In one such aspect, the disease is systemic lupus erythematosus and/or lupus nephritis.

In one aspect, there is provided a BLyS binding protein or a pharmaceutical composition comprising a BLyS binding protein as described herein for use in therapy. In a further aspect, the BLyS binding protein or the pharmaceutical composition comprising the BLyS binding protein as disclosed herein is for use in the treatment of diseases in which B cells play a pathogenic role. In a further aspect, the BLyS binding protein or the pharmaceutical composition comprising the BLyS binding protein as disclosed herein is for use in the treatment of an autoimmune disease, a cancer, a chronic kidney disease and/or a chronic inflammatory disease. In a further aspect, the BLyS binding protein or the pharmaceutical composition comprising the BLyS binding protein as disclosed herein is for use in the treatment of an autoimmune disease and/or a chronic inflammatory disease.

In a further aspect, the BLyS binding protein or the pharmaceutical composition comprising the BLyS binding protein as disclosed herein is for use in the treatment of systemic lupus erythematosus, lupus nephritis, systemic sclerosis, cutaneous lupus erythematosus, ANCA-associated vasculitis, systemic sclerosis-associated interstitial lung disease, connective tissue disease, and/or connective tissue disease-associated interstitial lung disease.

In one aspect, there is provided use of a BLyS binding protein or a pharmaceutical composition comprising a BLyS binding protein as described herein in the manufacture of a medicament for use in the treatment of disease.

### Definitions

The term “antibody” is used herein in the broadest sense to refer to molecules with an immunoglobulin-like domain (for example, IgG, IgM, IgA, IgD, or IgE) and includes monoclonal, recombinant, polyclonal, chimeric, human, humanized, multispecific antibodies, including bispecific antibodies, and heteroconjugate antibodies; a single variable domain (e.g., a domain antibody (DAB)), antigen binding antibody fragments, Fab, F(ab′)2, Fv, disulphide linked Fv, single chain Fv, disulphide-linked scFv, diabodies, TANDABS, etc. and modified versions of any of the foregoing. In some embodiments, an antibody comprises two heavy chains (HCs) (which are typically identical) and two light chains (LCs) (which are typically identical) linked by covalent disulphide bonds. This H2L2 structure can fold to form three functional domains: two fragment antigen binding regions (Fab regions) and a crystallisable fragment region (Fc region). A Fab region comprises a variable domain, which comprises variable heavy (VH) and variable light (VL) chains, at the amino-terminus, and a constant domain, which comprises a first domain of a constant heavy chain (CH) and a constant light chain (CL) at the carboxyl-terminus. An Fc region comprises two domains formed by dimerization of paired second and third domains (CH2 and CH3) of two constant heavy chains (CH).

An Fc region may elicit effector functions, for example, by binding to receptors on immune cells or by binding C1q, the first component of the classical complement pathway. Typically, the majority of antibodies in the serum belong to the IgG class; there are four isotypes of human IgG (IgG1, IgG2, IgG3, and IgG4), the sequences of which differ mainly in their hinge region. Antibodies provided herein can be fully human antibodies or humanized antibodies.

One or more of the antigen binding proteins described herein may be an antibody or an antigen binding fragment thereof. An antigen binding protein may be a humanized antibody or an antigen binding fragment thereof. An antigen binding protein may comprise one of, a plurality of, or all of: a humanized VH region, or a humanized Heavy Chain (HC) sequence; and/or a humanized VL region, or a humanized Light Chain (LC) sequence.

A “domain antibody” or “DAB” can be a human “single variable domain”. A single variable domain may be a human single variable domain but can also be a single variable domains from a non-human species such as rodent (for example, as in WO 00/29004), a nurse shark, or a camelid. Notably, camelid VHHs are immunoglobulin single variable domain polypeptides that are derived from camelid species, such as camel, llama, alpaca, dromedary, and guanaco, which produce heavy chain only antibodies that are naturally devoid of light chains. Such VHH domains may be humanized according to standard techniques available in the art, and such domains can be “single variable domains”.

An antigen binding fragment may be provided by means of arrangement of one or more CDRs on one or more non-antibody protein scaffolds.

“Protein Scaffold” as used herein includes, but is not limited to, an immunoglobulin (Ig) scaffold, for example, an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. A protein scaffold may be an Ig scaffold, for example, an IgG or IgA scaffold. An IgG scaffold may comprise some or all the domains of an antibody (i.e., CH1, CH2, CH3, VH, VL). An antigen binding protein may comprise an IgG scaffold selected from IgG1, IgG2, IgG3, IgG4, or IgG4PE. For example, a scaffold may be IgG1. A scaffold may consist of, or comprise, an Fc region of an antibody, or a fragment thereof. A protein scaffold may be a derivative of a scaffold selected from the group consisting of CTLA-4, lipocalin, Protein A derived molecules such as Z-domain of Protein A (Affibody, SpA), Adomain (Avimer/Maxibody); heat shock proteins such as GroEI and GroES; transferrin (trans-body); ankyrin repeat protein (DARPin); peptide aptamer; C-type lectin domain (Tetranectin); human crystallin and human ubiquitin (affilins); PDZ domains; scorpion toxin kunitz type domains of human protease inhibitors; and fibronectin/adnectin; which has been subjected to protein engineering in order to obtain binding to an antigen, such as BLyS, other than a natural ligand.

The term multi-specific antigen binding protein refers to an antigen binding protein that comprises at least two different antigen binding sites. Each of these antigen-binding sites can be capable of binding to a different epitope than another of the antigen-binding sites; the antigen binding sites can be present on the same antigen or on different antigens. A multi-specific antigen binding protein may have specificity for more than one antigen, for example, two antigens, or three antigens, or four antigens. A multi-specific antigen binding protein having specificity for two antigens can be referred to as a bispecific antigen binding protein. A bispecific antigen binding protein (i.e., a bispecific) can be classified as having a symmetric or asymmetric architecture. A bispecific antigen binding protein can be a bispecific antibody, for example, a HET mAb. A bispecific may have an Fc region or may be fragment-based (lacking an Fc region). A fragment based bispecific can combine multiple antigen-binding fragments in one molecule without an Fc region or with a portion of an Fc region, e.g., Fab-scFv, Fab-scFv2, orthogonal Fab-Fab, Fab-Fv, tandem scFc (e.g., BiTE and BiKE molecules), Diabody, DART, TandAb, scDiabody, tandem dAb, etc. A symmetric format can combine multiple binding specificities in a single polypeptide chain or single HL pair. Examples can include an Fc-fusion protein(s) of a fragment-based format or a format whereby one or more antibody fragments are fused to an antibody molecule or other antigen binding protein. Examples of symmetric formats may include DVD-Ig, TVD-Ig, CODV-Ig, (scFv) 4-Fc, IgG-(scFv) 2, Tetravalent DART-Fc, F (ab) 4CrossMab, IgG-HC-scFv, IgG-LC-scFv, mAb-dAb, etc. An asymmetric format can retain as closely as possible the native architecture of a natural antibody by forcing correct HL chain pairing and/or promoting H chain heterodimerization during the co-expression of three (if common heavy or light chains are used) or four polypeptide chains, e.g., Triomab, asymmetric reengineering technology immunoglobulin (ART-Ig), CrossMab, Biclonics common light chain, ZW1 common light chain, DuoBody and knobs into holes (KiH), DuetMab, κλ body, Xmab, YBODY, HET-mAb, HET-Fab, DART-Fc, SEEDbody, mouse/rat chimeric IgG. Bispecific formats can also include an antibody fused to a non-Ig scaffold, such as Affimabs, Fynomabs, Zybodies, Anticalin-IgG fusions, and ImmTAC.

One or more antigen binding proteins described herein may show cross-reactivity between human BLyS and BLyS from another species, such as cynomolgus BLYS or rhesus BLyS. An antigen binding protein described herein may specifically bind human BLyS and cynomolgus BLyS. Such cross-reactivity can be exploited during preclinical research, e.g., in one or more non-human primate systems such as rhesus monkey or cynomolgus monkey. Such preclinical research can be performed before the antigen binding protein is tested in humans. Such cross-reactivity can be exploited to make one or more side-by-side comparisons of using an antigen binding protein herein. Cross reactivity between other species that can be used in disease models, such as dog or another monkey. Optionally, the binding affinity of the antigen binding protein for at least cynomolgus BLyS and the binding affinity for human BLyS differ by no more than a factor of 2, 5, 10, 50, or 100. The amino acid sequence of human BLyS is set out in UniProtKB reference Q9Y275.

Affinity, also referred to as “binding affinity”, is the strength of binding at a single interaction site, i.e., of one molecule, e.g., an antigen binding protein of the invention, to another molecule, e.g., BLyS, at a single binding site. The binding affinity of an antigen binding protein to its target may be determined by equilibrium methods (e.g., using enzyme-linked immunosorbent assay (ELISA) or radioimmunoassay (RIA)), or kinetics (e.g., BIACORE analysis). For example, SPR methods may be used to measure binding affinity. Avidity, also referred to as functional affinity, is the cumulative strength of binding at multiple interaction sites, e.g., the sum total of the strength of binding of two molecules (or more, e.g., in the case of a bispecific or multispecific molecule) to one another at multiple sites, e.g., taking into account the valency of the interaction. The equilibrium dissociation constant (KD) of an antigen binding protein-BLyS interaction may be 1000 pM or less. Alternatively, The KD may be between 1000 pM and 500 pM; between 500 pM and 250 pM; or between 250 and 100 pM. The KD may be between 1 pM and 10 pM; between 10 pM and 20 pM; or between 10 and 50 pM; or the KD may be between 5 pM and 80 pM. In one embodiment, the BLyS binding protein as disclosed herein binds to BLyS, for example, to human BLyS with a KD of less than or equal to 150 pM, less than or equal to 140 pM, less than or equal to 130 pM, less than or equal to 120 pM, less than or equal to 110 pM, less than or equal to 100 pM, less than or equal to 90 pM, less than or equal to 80 pM, less than or equal to 70 pM, less than or equal to 60 pM, less than or equal to 50 pM, or less than or equal to 40 pM (for example, 39 pM). For antigen binding proteins herein, a smaller KD numerical value corresponds with stronger binding to an antigen such as BLyS. The reciprocal of KD (i.e., 1/KD) is the equilibrium association constant (KA) and can be expressed as M⁻¹. For antigen binding proteins herein, a larger KA numerical value corresponds with stronger binding to an antigen such as BLyS. The dissociation rate constant (kd) or “off-rate” describes the stability of the antigen binding protein-antigen (e.g., BLyS) complex, i.e., the fraction of complexes that decay per second. For example, a kd of 0.01 s⁻¹ equates to 1% of the complexes decaying per second. It will be appreciated that the antigen binding proteins exemplified in the present application have exceptionally high binding affinity, for example, a KD of 39 pM.

As used herein, “isolated” can be used in reference to a molecule, such as an antigen binding protein, antigen, nucleic acid, peptide, or another molecule that is removed from the environment in which it is produced, from an environment in which it may be found in nature, or from another environment. An antigen binding protein described herein, for example, an anti-BLyS antibody, may be encoded by one or more isolated nucleic acid sequences. Production of a BLyS binding protein, such as an antibody, may be achieved in a cell or living organism by delivering exogenous isolated nucleic acids encoding the BLyS binding protein, for example, a heavy chain and a light chain of an antibody. A subject in need may be delivered one or more nucleic acids encoding an antigen binding protein provided herein, such as a heavy chain and a light chain of an anti-BLyS antibody. The heavy chain and the light chain of the antibody may be delivered by the same or separate nucleic acids. The nucleic acids may be DNA or RNA. The nucleic acids may be mRNA. The nucleic acid coding for the BLyS binding proteins may be modified or unmodified. The nucleic acids coding for the BLyS binding proteins may comprise at least one chemical modification. The nucleic acids encoding the BLyS binding protein may be delivered to the subject naked (i.e., without an encapsulating particle) or packaged (i.e., encapsulated in liposomes or polymer-based vehicles). Nucleic acids (e.g., mRNAs) can be modified to enhance stability by including one or more chemical modifications. Such chemical modifications include, but are not limited to, a modified nucleotide, a modified sugar backbone, a cap structure, a poly A tail, or a 5′ and/or a 3′ untranslated region. Also provided herein is a method of producing a BLyS binding protein in a cell, tissue, or organism comprising contacting said cell, tissue, or organism with a composition comprising an isolated nucleic acid comprising at least one chemical modification and which encodes the BLyS binding protein. Also provided herein is a method of producing a BLyS binding protein in a cell, tissue, or organism comprising contacting said cell, tissue, or organism with a composition comprising a polynucleotide comprising at least one chemical modification and which encodes a BLyS binding protein. Also provided herein are expression vectors, wherein an expression vector can be an isolated nucleic acid which can be used to introduce a nucleic acid of interest into a cell, such as a eukaryotic cell or prokaryotic cell, or a cell free expression system where the nucleic acid sequence of interest is expressed as a peptide chain such as a protein. A nucleic acid of interest can comprise a nucleic acid sequence of an antigen binding protein provide herein or a fragment thereof. Such expression vectors may be, for example, cosmids, plasmids, viral sequences, transposons, and linear nucleic acids comprising a nucleic acid of interest. Once the expression vector is introduced into a cell or cell free expression system (e.g., reticulocyte lysate) the protein encoded by the nucleic acid of interest is produced by the transcription/translation machinery. Expression vectors within the scope of the disclosure may provide necessary elements for eukaryotic or prokaryotic expression and include viral promoter driven vectors, such as CMV promoter driven vectors, e.g., pcDNA3.1, pCEP4, and their derivatives, Baculovirus expression vectors, Drosophila expression vectors, and expression vectors that are driven by mammalian gene promoters, such as human Ig gene promoters. Other examples include prokaryotic expression vectors, such as T7 promoter driven vectors, e.g., pET41, lactose promoter driven vectors, and arabinose gene promoter driven vectors. Those of ordinary skill in the art will recognise many other suitable expression vectors and expression systems.

Also provided herein are recombinant host cells. The term “recombinant host cell” as used herein refers to a cell that comprises a nucleic acid sequence of interest that was isolated prior to its introduction into the cell. For example, the nucleic acid sequence of interest may be in an expression vector while the cell may be prokaryotic or eukaryotic. Exemplary eukaryotic cells are mammalian cells, such as but not limited to, COS-1, COS-7, HEK293, BHK21, CHO, BSC-1, HepG2, 653, SP2/0, NS0, 293, HeLa, myeloma, lymphoma cells, or any derivative thereof. The eukaryotic cell may be HEK293, NS0, SP2/0, or CHO cell. E. coli is an exemplary prokaryotic cell. A recombinant cell according to the disclosure may be generated by transfection, cell fusion, immortalisation, or other procedures well known in the art. A nucleic acid of interest, such as an expression vector, transfected into a cell may be extrachromosomal or stably integrated into the chromosome of the cell.

Also provided herein are complementarity determining region (CDR) amino acid sequences of antigen binding proteins. These are the hypervariable regions of antigen binding proteins herein, such as immunoglobulin heavy and light chains. Typically, there are three heavy chain and three light chain CDRs (or CDR regions) in the variable portion of an antigen binding protein such as an immunoglobulin. Thus, “CDRs” as used herein can refer to three heavy chain CDRs of an antigen binding protein, three light chain CDRs of an antigen binding protein, all heavy and light chain CDRs of an antigen binding protein, or at least two CDRs of an antigen binding protein. Throughout this specification, amino acid residues in variable domain sequences and variable domain regions within full-length antigen binding sequences, e.g., within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4th Ed., U.S. Department of Health and Human Services, National Institutes of Health (1987). There are alternative numbering conventions for amino acid residues in variable domain sequences and full-length antibody sequences. Throughout this specification, amino acid residues in Fc regions, in antibody sequences or full-length antigen binding protein sequences, are numbered according to the EU index numbering convention. There are also alternative numbering conventions for CDR sequences, for example, those set out in Chothia et al. (1989) Nature 342:877-883. The structure and protein folding of the antigen binding protein may mean that other residues are considered part of the CDR sequence and would be understood to be so by a skilled person. Other numbering conventions for CDR sequences available to a skilled person include “AbM” (University of Bath) and “contact” (University College London) methods. The CDR regions can be defined by any numbering convention, for example, the Kabat, Chothia, AbM and contact conventions.

Table A below represents one definition using each numbering convention for CDRs, or binding unit, provided herein. The Kabat numbering scheme is used in the specification to number the variable domain amino acid sequence. It should be noted that CDR definitions can vary depending on the individual publication used.

TABLE A
CDR definitions

| | | | | | Minimum |
|---|---|---|---|---|---|
| | Kabat | Chothia | AbM | Contact | Binding |
| | CDR | CDR | CDR | CDR | Unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Accordingly, in one aspect, a BLyS binding protein is provided, which comprises any one or a combination of the following CDRs: CDRH1, CDRH2, and CDRH3 from SEQ ID NO:7 and/or CDRL1, CDRL2, and CDRL3 from SEQ ID NO:8. In a further aspect, a BLyS binding protein is provided, which comprises any one or a combination of the following CDRs: CDRH1, CDRH2, and CDRH3 from SEQ ID NO:21 and/or CDRL1, CDRL2, and CDRL3 from SEQ ID NO:22.

CDRs of a BLyS binding protein provided herein can be modified by one or by more than one amino acid substitution, deletion, or addition, wherein the variant BLyS binding protein substantially retains the biological characteristics of the unmodified protein, such as inhibiting the binding of BLyS to its receptors. It will be appreciated that each of CDR H1, H2, H3, L1, L2, L3 may be modified alone or in combination with any other CDR, in any permutation or combination. A CDR may be modified by the substitution, deletion, or addition of up to 3 amino acids, for example, 1 or 2 amino acids, for example, 1 amino acid. Each modification of a CDR, VH, VL, or other protein provided herein can be a conservative substitution. A modification can be a conservative substitution. Examples of conservative substitutions by side chain type include: Hydrophobic (e.g., Met, Ala, Val, Leu, Ile); Neutral hydrophilic (e.g., Cys, Ser, Thr); Acidic (e.g., Asp, Glu); Basic (e.g., Asn, Gln, His, Lys, Arg); Residues that influence chain orientation (e.g., Gly, Pro); and Aromatic (e.g., Trp, Tyr, Phe). For example, in a variant CDR, one or more flanking residues that comprise the CDR as part of alternative definition(s) e.g., Kabat or Chothia, may be substituted with a conservative amino acid residue. Such antigen binding proteins comprising variant CDRs as described above may be referred to herein as “functional CDR variants”.

A BLyS binding protein can be an antagonist, such as an antagonist antibody. An antagonist can comprise an epitope binding protein, such as an antibody or fragment thereof, that is capable of fully or partially inhibiting the biological activity of the antigen to which it binds, for example, by fully or partially blocking binding of the antigen to a receptor or by neutralising activity, such as signalling, which can be initiated by a biological activity of the antigen.

A BLyS binding protein herein can be neutralising. “Neutralise” refers to a reduction or elimination of the biological activity of the antigen (e.g., BLyS) in the presence of a BLyS binding protein as described herein, in comparison to the biological activity of the antigen in the absence of the BLyS binding protein, in vitro or in vivo. Neutralisation may be due to one or more of blocking BLyS binding to its receptor, preventing BLyS from activating its receptor, down regulating BLYS or its receptor, or affecting effector functionality. Neutralisation may be determined or measured using one or more assays, for example, as described herein. For example, the blocking assay methods described in Examples 6 and 7 may be used to assess the neutralising capability of an antigen binding protein herein. The effect of a BLyS binding protein on the interaction between BLyS and its receptors such as BAFFR may be partial or total. A neutralising BLyS binding protein may neutralize the activity of BLyS-BAFFR interactions (e.g., binding) by at least 20%, at least 30%, at least 40%, at least 50%, at least 55%, at least 60%, at least 65%, at least 70%, at least 75%, at least 80%, at least 82%, at least 84%, at least 86%, at least 88%, at least 90%, at least 92%, at least 94%, at least 95%, at least 96%, at least 97%, at least 98%, at least 99%, or 100% relative to BLyS-BAFFR interactions in the absence of the BLyS binding protein. BLyS binding proteins described herein may inhibit the interaction of human BLyS and human BAFFR by 70% or more, 75% or more, 80% or more, 85% or more, 90% or more, or 95% or more. BLyS binding proteins described herein may inhibit the binding of soluble BLyS to BAFFR by 70% or more, 75% or more, 80% or more, 85% or more, 90% or more, or 95% or more.

“Percent identity” or “% identity” between a query nucleic acid sequence and a subject nucleic acid sequence is the “Identities” value, expressed as a percentage, that is calculated using a suitable algorithm (e.g., BLASTN, FASTA, Needleman-Wunsch, Smith-Waterman, LALIGN, or GenePAST/KERR) or software (e.g., DNASTAR Lasergene, GenomeQuest, EMBOSS needle or EMBOSS infoalign), over the length of the query sequence after alignment, such as a pair-wise global sequence alignment, has been performed using a suitable algorithm (e.g., Needleman-Wunsch or GenePAST/KERR) or software (e.g., DNASTAR Lasergene or GenePAST/KERR). A query nucleic acid sequence may be described by a nucleic acid sequence disclosed herein, in particular, in one or more of the claims or clauses.

“Percent identity” or “% identity” between a query amino acid sequence and a subject amino acid sequence is the “Identities” value, expressed as a percentage, that is calculated using a suitable algorithm (e.g., BLASTP, FASTA, Needleman-Wunsch, Smith-Waterman, LALIGN, or GenePAST/KERR) or software (e.g., DNASTAR Lasergene, GenomeQuest, EMBOSS needle or EMBOSS infoalign), over the entire length of the query sequence after a pair-wise global sequence alignment has been performed using a suitable algorithm (e.g., Needleman-Wunsch or GenePAST/KERR) or software (e.g., DNASTAR Lasergene or GenePAST/KERR). A query amino acid sequence may be described by an amino acid sequence disclosed herein, in particular, in one or more of the claims or clauses.

A query sequence may be 100% identical to a subject sequence, or it may include up to a certain integer number of amino acid or nucleotide alterations as compared to the subject sequence such that the % identity is less than 100%. For example, a query sequence can be at least 50%, at least 60%, at least 70%, at least 75%, at least 80%, at least 85%, at least 90%, at least 95%, at least 96%, at least 97%, at least 98%, or at least 99% identical to a subject sequence. In the case of nucleic acid sequences, such alterations can comprise at least one nucleotide residue deletion, substitution or insertion, wherein said alterations may occur at the 5′- or 3′-terminal positions of a query sequence or at one or more positions between those terminal positions, interspersed either individually among the nucleotide residues in the query sequence or in one or more contiguous groups within a query sequence. In the case of amino acid sequences, such alterations can comprise at least one amino acid residue deletion, substitution (including conservative and non-conservative substitutions), or insertion, wherein said alterations may occur at the amino- or carboxy-terminal positions of a query sequence, or at one or more positions between those terminal positions, interspersed either individually among the amino acid residues in a query sequence or in one or more contiguous groups within a query sequence. For antibody sequences, a % identity may be determined across the entire length of a query sequence, including the CDRs. A calculated % identity may exclude one or more or all of the CDRs. For example, all of the CDRs of an antibody may be 100% identical to a subject sequence, while a % identity in the remaining portion of the query sequence, e.g., the framework sequence can be less than 100%, such that that the CDR sequences are fixed and intact.

A BLyS binding protein provided herein can comprise a sequence that is a variant amino acid sequence. A nucleic acid sequence of a BLyS binding protein provided herein can comprise a variant nucleic acid sequence. A variant nucleic acid sequence herein can be of a BLyS binding protein provided herein or of a variant thereof. A VH or VL (or HC or LC) sequence may be a variant sequence of a VH or VL (or HC or LC) sequence provided herein. with up to 10 amino acid substitutions, additions, or deletions. Such a variant sequence may have 10, 9, 8, 7, 6, 5, 4, 3, 2, or 1 amino acid substitution(s), addition(s), or deletion(s). An HC sequence may be a variant sequence of an HC sequence provided herein with up to 40 amino acid substitutions, additions, or deletions. An HC variant sequence may have up to 35, up to 30, up to 25, up to 20, up to 15, or up to 10 amino acid substitutions, additions, or deletions. An HC variant sequence may have 10, 9, 8, 7, 6, 5, 4, 3, 2, or 1 amino acid substitutions, additions, or deletions. An LC sequence may be a variant sequence of an LC sequence provided herein with up to 15 amino acid substitutions, additions, or deletions. An LC variant sequence may have up to 15, up to 10, or up to 5 amino acid substitutions, additions, or deletions. An LC variant sequence may have 10, 9, 8, 7, 6, 5, 4, 3, 2, or 1 amino acid substitutions, additions, or deletions. A sequence variation may exclude one or more or all of the CDRs. For example, the CDRs portion of the VH or VL (or HC or LC) sequence can be free of a sequence variation, and the variation can be present in a non-CDR portion of a VH or VL (or HC or LC) sequence, i.e., such that the CDR sequences are intact.

In one aspect, the sequence variation can occur in any part of the heavy or light chain providing that the sequences of the CDRs (for example, SEQ ID NOs: 1-6 or 15-20) remain unchanged.

An antigen binding protein having a variant sequence can substantially retain the biological characteristics of an unmodified antigen binding protein, such as inhibiting binding of BLyS to BAFFR. A binding property (e.g., KD, Kd, or Ka) of a BLyS binding protein having a variant sequence can be substantially identical to an unmodified BLyS binding protein. A binding property (e.g., KD, Kd, or Ka) of a variant sequence can be at least 75%, at least 90%, at least 95%, or at least 99% identical to that of an unmodified BLyS binding protein.

Upon production of an antigen binding protein, such as an antibody in a host cell, post-translational modifications may occur. For example, a post-translational modification can comprise the cleavage of one or more leader sequences, the addition of one or more sugar moieties such as in a glycosylation pattern, non-enzymatic glycation, deamidation, oxidation, disulfide bond scrambling, and other cysteine variants, such as those comprising free sulfhydryls, racemized disulfides, thioethers and trisulfide bonds, isomerisation, C-terminal lysine clipping, and N-terminal glutamine cyclisation. The disclosure herein encompasses the use of antigen binding proteins that have been subjected to, or have undergone, one or more post-translational modifications.

Antigen binding proteins may be prepared by any of a number of conventional techniques. For example, an antigen binding protein may be purified from one or more cells that naturally express it (e.g., an antibody can be purified from a hybridoma that produces it) or produced in a recombinant expression system. More than one antigen binding protein can be expressed (and therefore purified) from such a natural cell or recombinant expression system. A number of different expression systems and purification regimes can be used to generate an antigen binding protein. Generally, host cells can be transformed with a recombinant expression vector encoding an antigen binding protein. The expression vector may be maintained by the host as a separate genetic element or integrated into the host chromosome depending on the expression system. A wide range of host cells can be employed, for example, Prokaryotes (including Gram negative or Gram-positive bacteria, for example, Escherichia coli, Bacilli sp., Pseudomonas sp., Corynebacterium sp.), Eukaryotes including yeast (for example, Saccharomyces cerevisiae, Pichia pastoris), fungi (for example, Aspergillus sp.), or higher Eukaryotes including insect cells and cell lines of mammalian origin (for example, CHO, NS0, PER.C6, HEK293, HeLa). A host cell may be an isolated host cell. A host cell is usually not part of a multicellular organism (e.g., plant or animal). For example, a host cell can be a single celled organism, or can be an individual cell of a multicellular organism that is separate from that organism. A host cell can be part of a multicellular organism, for example, a plant or animal. The host cell may be a non-human host cell. Appropriate cloning and expression vectors can be designed or engineered for use with bacterial, fungal, yeast, and mammalian host cells. A commercial vector can be obtained and engineered to be a vector for the expression of a BLyS binding protein provided herein. Cells of an expression system can be cultured under conditions that promote expression of the antigen binding protein using one or more of appropriate equipment, including a shake flask(s), a spinner flask(s), and a bioreactor(s). The antigen binding protein can be recovered by conventional protein purification procedures or modifications thereof. Protein purification procedures can comprise of a series of unit operations comprising one or more filtration or chromatographic processes, or a combination thereof, developed to selectively isolate and/or concentrate the antigen binding protein. The purified antigen binding protein may be formulated in a pharmaceutically acceptable composition.

Fc engineering methods can be applied to modify the functional or pharmacokinetic properties of an antigen binding protein comprising an Fc region, such as an antibody. Binding to Fcγ can promote ADCC activity; thus, ADCC activity may be altered by making mutations in the Fc region that can increase or decrease binding to Fcγ receptors. Binding to C1q can promote CDC activity; thus, CDC activity may be altered by making mutations in the Fc region that can increase or decrease binding to C1q receptor. Modifications to the glycosylation pattern of an antigen binding protein can also be made to change the effector function. The in vivo half-life of an antigen binding protein can be altered by making mutations that affect binding of the Fc region to the FcRn (Neonatal Fc Receptor).

The term “Effector Function” as used herein refers to one or more antigen binding protein (e.g., antibody) mediated effects, including but not limited to antibody-dependent cell-mediated cytotoxicity (ADCC), antibody-mediated complement activation including complement-dependent cytotoxicity (CDC), complement-dependent cell-mediated phagocytosis (CDCP), antibody dependent complement-mediated cell lysis (ADCML), and Fc-mediated phagocytosis or antibody-dependent cellular phagocytosis (ADCP). The interaction between the Fc region of an antigen binding protein comprising an Fc region, such as an antibody, and various Fc receptors (FcR), including FcγRI (CD64), FcγRII (CD32), FcγRIII (CD16), FcRn, C1q, and type II Fc receptors can mediate the effector functions of the antigen binding protein. Significant biological effects can be a consequence of effector functionality. The ability to mediate effector function can require binding of the antigen binding protein to an antigen. An antigen binding protein can mediate one of, a plurality of, or each effector function. Effector function can be assessed in a number of ways including, for example, by evaluating ADCC effector function of an antibody coated to target cells mediated by Natural Killer (NK) cells via FcγRIII, or monocytes/macrophages via FcγRI, or by evaluating CDC effector function of an antigen binding protein coated to target cells mediated by complement cascade via C1q. For example, an antigen binding protein described herein can be assessed for ADCC effector function in a Natural Killer cell assay. The effects of mutations, including mutations in the Fc region, on effector functions (including but not limited to FcRn binding, FcγRs and C1q binding, CDC, ADCML, ADCC, ADCP) can be assessed. An antigen binding protein can comprise one or more of such mutations. Some isotypes of human constant regions of antigen binding proteins provided herein, in particular, IgG4 and IgG2 isotypes, can partially or fully lack the functions of a) activation of complement by the classical pathway and b) ADCC. Various modifications to the heavy chain constant region of antigen binding proteins may be carried out to alter effector function depending on the desired effector property. IgG1 constant regions containing specific mutations that reduce binding to Fc receptors and reduce an effector function, such as ADCC and CDC, have been described. Provided herein are antigen binding proteins comprising a constant region such that the antigen binding protein has reduced effector function, such as reduced ADCC and/or CDC. The heavy chain constant region may comprise a naturally disabled constant region of an IgG2 or IgG4 isotype or a mutated IgG1 constant region. Non-limiting examples of suitable modifications are described in EP0307434. For example, a constant region can comprise substitution with alanine at positions 235 and 237 (EU index numbering), i.e., L235A and G237A (commonly referred to as “LAGA” mutations). Other examples can comprise substitution with alanine at positions 234 and 235 (EU index numbering), i.e., L234A and L235A (commonly referred to as “LALA” mutations). Additional examples can comprise substitution with alanine at positions 234, 235 and 237 (EU index numbering), i.e., L234A, L235A, and G237A (referred to as “LALAGA” mutations). Further examples, such as described in EP2691417 and U.S. Pat. No. 8,969,526, can comprise a substitution with glycine or arginine at position 329 (i.e., P329G or P329R), in combination with the LALA mutations (EU index numbering) for Fc regions including IgG1 Fc regions. Yet further examples can comprise a substitution with glycine or arginine at position 329 (i.e., P329G or P329R), in combination with a substitution with proline at position 228 and glutamic acid at position 235 (i.e., S228P and L235E) for Fc domains including IgG4 Fc regions (EU index numbering). Other mutations that can be employed to decrease effector function can include: (with reference to IgG1 unless otherwise noted): aglycosylated N297A or N297Q or N297G; L235E; IgG4: F234A/L235A; or chimeric IgG2/IgG4. IgG2 comprising H268Q/V309L/A330S/P331S or V234A/G237A/P238S/H268A/V309L/A330S/P331S substitutions can be employed to reduce FcγR and/or C1q binding. Other mutations that can be employed to decrease effector function can include L234F/L235E/P331S; a chimeric antibody created using the CH1 and hinge region from human IgG2 and the CH2 and CH3 regions from human IgG4; IgG2m4, based on the IgG2 isotype with four key amino acid residue changes derived from IgG4 (H268Q, V309L, A330S and P331S); IgG20 that contains V234A/G237A/P238S/H268A/V309L/A330S/P331S substitutions to eliminate affinity for Fcγ receptors and C1q complement protein; IgG2m4 (H268Q/V309L/A330S/P331S, changes to IgG4); IgG4 (S228P/L234A/L235A); huIgG1 L234A/L235A (AA); huIgG4 S228P/L234A/L235A; IgG1s (L234A/L235A/G237A/P238S/H268A/A330S/P331S); IgG4s1 (S228P/F234A/L235A/G237A/P238S); and IgG4s2 (S228P/F234A/L235A/DG236/G237A/P238S, wherein D denotes a deletion) (Tam et al., Antibodies 2017, 6 (3)). BLyS binding proteins described herein may comprise an IgG1 constant region comprising a N297G mutation. BLyS binding proteins described herein may comprise an Fc region comprising a N297G mutation.

Half-life (t½) refers to the time required for the serum concentration of an antigen binding protein to reach half of its original value (i.e., half of a determined serum concentration achieved post administration). The serum half-life of proteins can be measured by pharmacokinetic studies, for example, according to a method wherein radio-labelled protein is injected intravenously into mice and its plasma concentration is periodically measured as a function of time, for example, at about 3 minutes to about 72 hours after the injection. Other methods for pharmacokinetic analysis and determination of the half-life of a molecule can be envisioned by a skilled artisan. Long half-lives of IgG antibodies can be dependent on their binding to FcRn. Therefore, substitutions that increase the binding affinity of IgG to FcRn at pH 6.0 while maintaining the pH dependence of the interaction with target, for example, by engineering the constant region, may be employed. The in-vivo half-life of antigen binding proteins described herein may be altered, for example, by modification of a heavy chain constant domain or by modification of an FcRn binding domain therein. In adult mammals, FcRn, also known as the neonatal Fc receptor, can play a key role in maintaining serum antibody levels by acting as a protective receptor that binds and salvages antibodies of the IgG isotype from degradation. IgG molecules are endocytosed by endothelial cells and, if they bind to FcRn, are recycled out of the cells back into circulation. In contrast, IgG molecules that enter the cells and do not bind to FcRn and are targeted to the lysosomal pathway where they are degraded. FcRn may be involved in both antibody clearance and the transcytosis across tissues. Human IgG1 residues determined to interact directly with human FcRn include Ile253, Ser254, Lys288, Thr307, Gln311, Asn434 and His435. Mutations at any of these positions may be employed in an antigen binding protein herein, for example, to enable increased serum half-life and/or altered effector properties of antigen binding proteins provided herein. Antigen binding proteins described herein may have amino acid modifications that increase the affinity of the constant domain or fragment thereof for FcRn. Increasing the half-life (i.e., serum half-life) of therapeutic and diagnostic IgG antibodies and other bioactive molecules can provide benefits, which can include reducing the amount and/or frequency of dosing of these molecules. An antigen binding protein may comprise all or a portion (an FcRn binding portion) of an IgG constant domain having one or more of the following amino acid modifications. For example, with reference to IgG1, M252Y/S254T/T256E (commonly referred to as “YTE” mutations) and M428L/N434S (commonly referred to as “LS” mutations) can increase FcRn binding at pH 6.0. Half-life can also be increased by T250Q/M428L, V259I/V308F/M428L, N434A, and T307A/E380A/N434A mutations (with reference to IgG1 and Kabat numbering) in an antigen binding protein provided herein. Half-life and FcRn binding can also be increased by introducing H433K and N434F mutations (commonly referred to as “HN” or “NHance” mutations) (with reference to IgG1) (WO2006/130834) in an antigen binding protein provided herein. An antigen binding protein provided herein can comprise a variant Fc region with altered FcRn binding affinity, which can comprise an amino acid modification at any one or more of amino acid positions 238, 252, 253, 254, 255, 256, 265, 272, 286, 288, 303, 305, 307, 309, 311, 312, 317, 340, 356, 360, 362, 376, 378, 380, 386,388, 400, 413, 415, 424, 433, 434, 435, 436, 439, and 447 of the Fc region (EU index numbering). A modified IgG comprising an IgG constant domain of an antigen binding protein can comprise one or more amino acid modifications relative to a wild-type IgG constant domain, wherein the modified IgG can have an increased half-life compared to the half-life of an IgG having a wild-type IgG constant domain, and wherein the one or more amino acid modifications are at one or more of positions 251, 253, 255, 285-290, 308-314, 385-389, and 428-435. Alanine scanning mutagenesis can be employed to alter residues in the Fc region of an antigen binding protein provided herein, for example, a human IgG1 antibody, and thus alter binding to human FcRn. Positions that can effectively abrogate binding to FcRn when changed to alanine include I253, S254, H435, and Y436. Other positions can result in a less pronounced reduction in binding when mutated, for example, as follows: E233-G236, R255, K288, L309, S415, and H433. Several amino acid positions can exhibit improvement in FcRn binding when changed to alanine; notable among these include P238, T256, E272, V305, T307, Q311, D312, K317, D376, E380, E382, S424, and N434. Other amino acid positions can exhibit a slight improvement (D265, N286, V303, K360, Q362, and A378) or no change (S239, K246, K248, D249, M252, E258, T260, S267, H268, 10 S269, D270, K274, N276, Y278, D280, V282, E283, H285, T289, K290, R292, E293, E294, Q295, Y296, N297, S298, R301, N315, E318, K320, K322, S324, K326, A327, P329, P331, E333, K334, T335, S337, K338, K340, Q342, R344, E345, Q345, Q347, R356, M358, T359, K360, N361, Y373, S375, S383, N384, Q386, E388, N389, N390, K392, L398, S400, D401, K414, R416, Q418, Q419, N421, V422, E430, T437, K439, S440, S442, S444, and K447) in FcRn binding. In some antigen binding proteins provided herein, combination variants can yield a pronounced effect with respect to improved FcRn binding. For example, at least at pH 6.0, the E380A/N434A variant can display over 8-fold increase in binding to FcRn, relative to wild type IgG1, compared with a 2-fold increase for E380A and a 3.5-fold increase for N434A. The addition of T307A to this combination (E380A/N434A/T307A) can result in a 12-fold increase in binding relative to wild type IgG1. Antigen binding proteins described herein may comprise the E380A/N434A or E380A/N434A/T307A mutations and have increased binding to FcRn. In some antigen binding proteins provided herein, an improvement in IgG1-human FcRn complex stability can occur when substituting residues located in a band across the Fc-FcRn interface (e.g., M252, S254, T256, H433, N434, and Y436) or at the periphery (e.g., V308, L309, Q311, G385, Q386, P387, and N389). M252Y/S254T/T256E (“YTE”) and H433K/N434F/Y436H mutations can be combined to yield a high affinity to human FcRn. In some cases, such a combination can exhibit a 57-fold increase in affinity relative to the wild-type IgG1. The in vivo behaviour of such a mutated human IgG1 can exhibit an increase in serum half-life of up to at least 4-fold as compared to wild-type IgG1. Such an increase in serum half-life can be in the serum of a human, of a cynomolgus monkey, or of another subject. Also provided are antigen binding proteins with optimized binding to FcRn. An antigen binding protein may comprise at least one amino acid modification in the Fc region of said antigen binding protein, for example, wherein said modification is at an amino acid position selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446, and 447 of the Fc region. A BLyS binding protein can have 2, 3, 4, 5, 6, or more of such amino acid modifications in the Fc region of said BLyS binding protein. A BLyS binding protein can have an amino acid modification in the Fc region of said BLyS binding protein at another amino acid position, either instead of or in addition to an amino acid modification provided herein. A BLyS binding protein can have a modified half-life, either by introducing an FcRn-binding polypeptide into the antigen binding protein (for example, as in WO97/43316, U.S. Pat. Nos. 5,869,046, 5,747,035, WO96/32478, or WO91/14438), by fusing the antigen binding protein with antibodies whose FcRn-binding affinities are preserved, but affinities for other Fc receptors have been greatly reduced (for example, as in WO99/43713), or by fusing the antigen binding protein with FcRn binding domains of antibodies (WO00/09560, U.S. Pat. No. 4,703,039). Antigen binding proteins herein can comprise FcRn affinity enhanced Fc variants that can improve antibody cytotoxicity and/or half-life at pH 6.0. Such IgG variants can be produced as low fucosylated molecules. The resulting variants can result in increased serum persistence in hFcRn mice, as well as conserved enhanced ADCC. Exemplary variants can include (with reference to IgG1 and Kabat numbering): P230T/V303A/K322R/N389T/F404L/N434S; P228R/N434S; Q311R/K334R/Q342E/N434Y; C226G/Q386R/N434Y; T307P/N389T/N434Y; P230S/N434S; P230T/V305A/T307A/A378V/L398P/N434S; P230T/P387S/N434S; P230Q/E269D/N434S; N276S/A378V/N434S; T307A/N315D/A330V/382V/N389T/N434Y; T256N/A378V/S383N/N434Y; N315D/A330V/N361D/A387V/N434Y; V259I/N315D/M428L/N434Y; P230S/N315D/M428L/N434Y; F241L/V264E/T307P/A378V/H433R; T250A/N389K/N434Y; V305A/N315D/A330V/P395A/N434Y; V264E/Q386R/P396L/N434S/K439R; or E294del/T307P/N434Y (wherein ‘del’ indicates a deletion). Although substitutions in the constant region can significantly improve the functions of antigen binding proteins, such as therapeutic IgG antibodies, substitutions in the strictly conserved constant region can yield immunogenicity in humans, while substitution in the highly diverse variable region sequence can be less immunogenic.

The CDR residues of an antigen binding protein provided herein can be engineered to improve binding affinity of the antigen binding protein to the antigen (e.g., BLyS). The CDR and/or framework residues can be engineered to improve stability and decrease immunogenicity risk of an antigen binding protein provided herein. Improved affinity to the antigen (e.g., BLyS) can be achieved by affinity maturation using the phage or ribosome display of a randomized library. Improved stability of an antigen binding protein provided herein can be rationally obtained from sequence- or structure-based rational design. Decreasing the immunogenicity risk (deimmunization) of an antigen binding protein can be accomplished, for example, by one or more humanization methodologies and/or the removal of potential T-cell epitopes, which in some cases can be predicted using in silico technologies or anticipated by in vitro assays. Additionally, the variable region of an antigen binding protein can be engineered to lower the isoelectric point (pI) of the antibody. For an antigen binding protein, a longer half-life can be associated with such a reduced pI compared to wild type antigen binding proteins, in some cases despite comparable FcRn binding. A similar increase in half life can be achieved with other antigen binding proteins. Engineering or selecting antigen binding proteins with pH-dependent antigen binding can be used to modify antigen binding protein and/or antigen (e.g., BLyS) half-life. For example, the half-life of an IgG2 antibody can be shortened if antigen-mediated clearance mechanisms can degrade the antibody when bound to the antigen. Similarly, an antigen: antibody complex can impact the half-life of an antigen (e.g., BLyS), for example, by extending half-life by protecting the antigen from the typical degradation processes, or by shortening the half-life via antibody-mediated degradation (e.g., target-mediated drug disposition). BLyS binding proteins may have higher affinity for antigen at pH 7.4 as compared to endosomal pH (i.e., pH 5.5-6.0) such that the KD ratio at pH 5.5/pH 7.4 or at pH 6.0/pH 7.4 can be 2 or more. For example, to enhance the pharmacokinetic (PK) and pharmacodynamic (PD) properties of the antigen binding protein, pH-sensitive binding to the antigen binding protein can be achieved by introducing one or more histidine residues into one or more of the CDRs.

An antigen binding protein herein can comprise a recycling antibody engineered so that a single antibody molecule can bind to an antigen multiple times. A recycling antibody can dissociate from an antigen (e.g., BLyS) under acidic conditions within the cell. An antibody bound to a membrane-bound antigen can dissociate from the antigen in a pH-dependent manner. The dissociated antibody can then be recycled by FcRn while the antigen is transferred to lysosome and degraded. This mechanism can enable the antibody to bind to other antigens repeatedly in plasma and reduces the antibody clearance. An antigen binding protein can comprise a sweeping antibody, which can be engineered, for example, using a combination of variable region engineering (as described above “pH switch”) to enable the antibody to bind to an antigen (e.g., BLyS) in plasma and dissociate from the antigen in endosome (after which the antigen undergoes lysosomal degradation), and constant region engineering to increase the cellular uptake of the antibody-antigen complex into endosome mediated (e.g., through FcRn), FcγRIIb or potentially other surface receptors. A sweeping antibody can therapeutically target a soluble antigen (e.g., BLyS), enhancing elimination of the antigen from the circulation. In some cases, one or more of a panel of Fc variants with enhanced binding to FcRn, including M252Y, V308P, N434Y, with enhanced binding to FcRn that, in combination with pH-dependent binding to a target antigen (e.g., BLyS), can enhance clearance of a target antigen (e.g., BLyS) in comparison with a wild-type Fc region. FcγRIIb can be used to accelerate the uptake rate of antibody-antigen complexes into cells. A BLyS binding protein can comprise an FcγRIIb sweeping antibody, or an Fc region thereof, in which the Fc region of a pH-dependent antibody can be engineered to selectively increase human FcγRIIb binding to enhance the uptake rate of antibody-antigen complexes. This inhibitory receptor can mediate the uptake of antibody-antigen complexes into liver endothelial cells (LSEC). Therefore, mediation of the uptake of an antigen binding protein (e.g., antibody): antigen complex into a cell by FcγRIIb (e.g., human FcγRIIb) can reduce antigen (e.g., BLyS) concentration in the circulation. A BLyS binding protein can comprise an Fc variant (v12) comprising the following mutations: E233D/G237D/P238D/H268D/P271G/A330R and can have selectively increased binding affinity to human FcγRIIb. In some cases, such a v12 variant can accelerate the clearance of antigen (e.g., BLyS) over that of a pH-dependent antibody with wildtype hIgG1 while maintaining comparable pharmacokinetics.

Provided herein are pharmaceutical compositions, wherein a pharmaceutical composition can comprise an antigen binding protein provided herein. Pharmaceutical compositions herein can be for use in the treatment of diseases, including human diseases, described herein. The pharmaceutical composition may comprise an antigen binding protein, optionally in combination with one or more pharmaceutically acceptable carriers and/or excipients. Such compositions can comprise a pharmaceutically acceptable carrier, for example, as is known by and called for by current pharmaceutical practice. Pharmaceutical compositions may be administered by injection or continuous infusion via a route, which can include, for example, intravenous, intraperitoneal, intradermal, subcutaneous, intramuscular, intraocular, intraportal, or another route. A pharmaceutical composition may be suitable for intravenous administration. A pharmaceutical composition may be suitable for subcutaneous administration. Pharmaceutical compositions may be suitable for topical administration (which can include, but is not limited to, epicutaneous, intranasal, or ocular administration), inhalational administration, or enteral administration (which can include, but is not limited to, oral, vaginal, or rectal administration). Pharmaceutical compositions provided herein can comprise an effective amount of an antigen binding protein, such as a BLyS binding protein.

A pharmaceutical composition may be included in a kit containing the antigen binding protein together with other medicaments and/or with instructions for use. For convenience, the kit may comprise the reagents in predetermined amounts with instructions for use. The kit may also include one or more devices, such as a syringe, a needle, a length of tubing, or another device, which can be used for administration of the pharmaceutical composition. The terms “individual”, “subject”, and “patient” are used herein interchangeably. The subject may be an animal. The subject may be a mammal, such as a primate, for example, a marmoset or monkey. The subject may be a human.

The antigen binding protein described herein may also be used in methods of treatment. It will be appreciated by those skilled in the art that references herein to treatment refer to the treatment of established conditions. However, antigen binding proteins disclosed herein may, depending on the condition, also be useful in the prevention of certain diseases. The antigen binding protein described herein is used in an effective amount for therapeutic, prophylactic, or preventative treatment. A therapeutically effective amount of the antigen binding protein described herein is an amount effective to ameliorate or reduce one or more symptoms of, or to prevent or cure, the disease. The term “prevention” refers to avoidance of the stated disease in a subject who is not suffering from the stated disease.

Provided herein are methods of treating diseases in which B cells play a pathogenic role. The phrase “diseases in which B cells play a pathogenic role” as used herein refers to diseases in which the dysregulation of B cell activity contributes to the development, maintenance and/or progression of a disease state.

In one aspect, the diseases are autoimmune diseases or cancer. The term “autoimmune disease” as used herein refers to a group of conditions that result from dysregulation of the immune system and the breakdown of immunological tolerance. The dysregulation of BLyS has been implicated in the pathogenesis of autoimmune disease. Without wishing to be bound by any particular theory, elevated levels of BLyS are thought to promote the survival and expansion of autoreactive B cells, which can produce autoreactive antibodies and contribute to autoimmune disease development, maintenance and/or progression.

In one aspect, the disease is a chronic kidney disease or chronic inflammatory disease. The term “chronic inflammatory disease” as used herein refers to a long-term condition associated with inflammation. Without wishing to be bound by any particular theory, it has been observed that B cells are present in inflamed tissue.

Diseases in which B cells play a pathogenic role may encompass autoimmune diseases, such as systemic lupus erythematosus, lupus nephritis, systemic sclerosis, cutaneous lupus erythematosus, ANCA-associated vasculitis, systemic sclerosis-associated interstitial lung disease, connective tissue disease, and/or connective tissue disease-associated interstitial lung disease.

Provided herein are methods of treating such diseases in a subject in need thereof comprising administering to said subject a therapeutically effective amount of the BLyS binding protein or pharmaceutical composition as defined herein. The subject may be an animal or a human. The subject may be a human. The BLyS binding proteins or pharmaceutical compositions described herein are provided for use in therapy. BLyS binding proteins or pharmaceutical compositions are provided for use in the treatment of autoimmune disease or cancer, or a chronic kidney disease or a chronic inflammatory disease. A BLyS binding protein is provided for use in the treatment of autoimmune disease or cancer, or a chronic kidney disease or a chronic inflammatory disease, wherein the BLyS binding protein comprises a VH region comprising SEQ ID NO:7 and a VL region comprising SEQ ID NO:8. A BLyS binding protein is provided for use in the treatment of autoimmune disease or cancer, or a chronic kidney disease or a chronic inflammatory disease, wherein the BLyS binding protein comprises an HC sequence comprising SEQ ID NO:9 and an LC sequence comprising SEQ ID NO:10. In one aspect, the BLyS binding protein provided is for use in the treatment of an autoimmune disease and/or a chronic inflammatory disease.

Also provided is the use of a BLyS binding protein in the manufacture of a medicament for the treatment of autoimmune disease or cancer, or a chronic kidney disease or a chronic inflammatory disease. Also provided is the use of a BLyS binding protein in the manufacture of a medicament for the treatment of autoimmune disease or cancer, or a chronic kidney disease or a chronic inflammatory disease, wherein the BLyS binding protein comprises a VH region comprising SEQ ID NO:7 and a VL region comprising SEQ ID NO: 8. Also provided is the use of a BLyS binding protein in the manufacture of a medicament for the treatment of autoimmune disease or cancer, or a chronic kidney disease or a chronic inflammatory disease, wherein the BLyS binding protein comprises an HC sequence comprising SEQ ID NO:9 and an LC sequence comprising SEQ ID NO:10. In one aspect, the use of the BLyS binding protein is in the manufacture of a medicament for the treatment of an autoimmune disease and/or a chronic inflammatory disease.

Also provided is a method for treatment of autoimmune disease or cancer, or a chronic kidney disease or a chronic inflammatory disease in a subject in need thereof comprising administering to said subject a therapeutically effective amount of a BLyS binding protein or a pharmaceutical composition described herein. Also provided is a method for treatment of autoimmune disease or cancer, or a chronic kidney disease or a chronic inflammatory disease, in a subject in need thereof comprising administering to said subject a therapeutically effective amount of a BLyS binding protein, wherein the BLyS binding protein comprises a VH region comprising SEQ ID NO:7 and a VL region comprising SEQ ID NO:8. Also provided is a method for treatment of autoimmune disease or cancer, or a chronic kidney disease or a chronic inflammatory disease, in a subject in need thereof comprising administering to said subject a therapeutically effective amount of a BLyS binding protein, wherein the BLyS binding protein comprises an HC sequence comprising SEQ ID NO:9 and an LC sequence comprising SEQ ID NO: 10. In one aspect, the method of treatment is of an autoimmune disease and/or a chronic inflammatory disease in the subject in need thereof.

A BLyS binding protein is provided for use in the treatment of autoimmune disease or cancer, or a chronic kidney disease or a chronic inflammatory disease wherein the BLyS binding protein comprises a VH region comprising SEQ ID NO:21 and a VL region comprising SEQ ID NO:22. A BLyS binding protein is provided for use in the treatment of autoimmune disease or cancer, or a chronic kidney disease or a chronic inflammatory disease, wherein the BLyS binding protein comprises a heavy chain sequence comprising SEQ ID NO: 23 and a light chain sequence comprising SEQ ID NO:24. In one aspect, the BLyS binding protein provided is for use in the treatment of an autoimmune disease and/or a chronic inflammatory disease.

Also provided is the use of a BLyS binding protein in the manufacture of a medicament for the treatment of autoimmune disease or cancer, or a chronic kidney disease or a chronic inflammatory disease. Also provided is the use of a BLyS binding protein in the manufacture of a medicament for the treatment of autoimmune disease or cancer, or a chronic kidney disease or a chronic inflammatory disease, wherein the BLyS binding protein comprises a VH region comprising SEQ ID NO:21 and a VL region comprising SEQ ID NO:22. Also provided is the use of a BLyS binding protein in the manufacture of a medicament for the treatment of autoimmune disease or cancer, or a chronic kidney disease or a chronic inflammatory disease, wherein the BLyS binding protein comprises a heavy chain sequence comprising SEQ ID NO:23 and a light chain sequence comprising SEQ ID NO:24. In one aspect, the use of the BLyS binding protein is in the manufacture of a medicament for the treatment of an autoimmune disease and/or a chronic inflammatory disease.

Also provided is a method for treatment of autoimmune disease or cancer, or a chronic kidney disease or a chronic inflammatory disease, in a subject in need thereof comprising administering to said subject a therapeutically effective amount of a BLyS binding protein or a pharmaceutical composition described herein. Also provided is a method for treatment of autoimmune disease or cancer, or a chronic kidney disease or a chronic inflammatory disease, in a subject in need thereof comprising administering to said subject a therapeutically effective amount of a BLyS binding protein, wherein the BLyS binding protein comprises a VH region comprising SEQ ID NO:21 and a VL region comprising SEQ ID NO:22. Also provided is a method for treatment of autoimmune disease or cancer, or a chronic kidney disease or a chronic inflammatory disease, in a subject in need thereof comprising administering to said subject a therapeutically effective amount of a BLyS binding protein, wherein the BLyS binding protein comprises a heavy chain sequence comprising SEQ ID NO: 23 and a light chain sequence comprising SEQ ID NO:24. In one aspect, the method of treatment is of an autoimmune disease and/or a chronic inflammatory disease in the subject in need thereof.

Diseases in which B cells play a pathogenic role include, but are not limited to, autoimmune diseases, cancer, chronic kidney disease, and chronic inflammatory disease. For example, diseases such as lupus, systemic lupus erythematosus, lupus nephritis, systemic sclerosis, cutaneous lupus erythematosus, ANCA-associated vasculitis, systemic sclerosis-associated interstitial lung disease, connective tissue disease, and/or connective tissue disease-associated interstitial lung disease.

Embodiments are further described in the subsequent numbered clauses:

1. A BLyS binding protein comprising one or more CDRs selected from CDRH1 of SEQ ID NO:1, CDRH2 of SEQ ID NO:2, CDRH3 of SEQ ID NO:3, CDRL1 of SEQ ID NO:4, CDRL2 of SEQ ID NO:5, and CDRL3 of SEQ ID NO:6.

2. The BLyS binding protein according to clause 1, comprising CDRH1 of SEQ ID NO:1, CDRH2 of SEQ ID NO:2, CDRH3 of SEQ ID NO:3, CDRL1 of SEQ ID NO:4, CDRL2 of SEQ ID NO:5, and CDRL3 of SEQ ID NO:6.

3. The BLyS binding protein according to clause 1 or 2, comprising a VH region at least 90% identical to the sequence of SEQ ID NO:7 and/or a VL region at least 90% identical to the sequence of SEQ ID NO:8.

4. The BLyS binding protein according to clause 3, comprising a VH region of SEQ ID NO:7 and a VL region of SEQ ID NO:8.

5. The BLyS binding protein according to any preceding clause, wherein the BLyS binding protein comprises an Fc region.

6. The BLyS binding protein according to clause 5, wherein the BLyS binding protein is a monoclonal antibody.

7. The BLyS binding protein according to clause 5, wherein the BLyS monoclonal antibody is an IgG1 antibody.

8. The BLyS binding protein according to any one of clauses 5 to 7, wherein the Fc region confers increased half-life relative to a wild-type Fc region.

9. The BLyS binding protein according to any one of clauses 5 to 8, wherein the BLyS binding protein comprises a heavy chain Fc domain having a tyrosine residue at position 252, a threonine residue at position 254, and a glutamic acid residue at position 256.

10. The BLyS binding protein according to any preceding clause, wherein the BLyS binding protein inhibits the binding of human BLyS to its receptor.

11. The BLyS binding protein according to clause 10, wherein the antibody inhibits the binding of human BLyS to BAFFR with an IC50 of 50 pM or less.

12. The BLyS binding protein according to any preceding clause, wherein the BLyS binding protein comprises a heavy chain of SEQ ID NO:9 and a light chain of SEQ ID NO:10.

13. A nucleic acid sequence which encodes one or both of the heavy chain and light chain of the BLyS binding protein as defined in any one of the preceding clauses.

14. The nucleic acid sequence according to clause 13, wherein the sequence comprises SEQ ID NO: 11 encoding the heavy chain and/or SEQ ID NO: 12 encoding the light chain.

15. An expression vector comprising the nucleic acid sequence(s) as defined in clauses 13 or 14.

16. A recombinant host cell comprising the nucleic acid sequence(s) as defined in clauses 13 or 14, or the expression vector(s) as defined in clause 15.

17. A method for the production of a BLyS binding protein, which method comprises culturing the host cell as defined in clause 16 under conditions suitable for expression of said nucleic acid sequence(s) or vector(s), whereby a polypeptide comprising the BLyS binding protein is produced.

18. The BLyS binding protein produced by the method of clause 17.

19. A cell line engineered to express the BLyS binding protein of any one of clauses 1 to 12 or 18.

20. A pharmaceutical composition comprising the BLyS binding protein as defined in any one of clauses 1 to 12 or clause 18 and a pharmaceutically acceptable excipient.

21. A method for the treatment of a disease where B cells play a pathogenic role comprising administering a therapeutically effective amount of the BLyS binding protein as defined in any one of clauses 1 to 12 or clause 18, or the pharmaceutical composition as defined in clause 20.

22. A method for the treatment of systemic lupus erythematosus, lupus nephritis, systemic sclerosis, cutaneous lupus erythematosus, anti-neutrophil cytoplasmic antibody (ANCA)-associated vasculitis, systemic sclerosis-associated interstitial lung disease, connective tissue disease, and/or connective tissue disease-associated interstitial lung disease in a human in need thereof comprising administering to said human a therapeutically effective amount of the BLyS antibody of clause 7 or clause 9.

23. A method of treatment according to clause 22, wherein the disease is systemic lupus erythematosus and/or lupus nephritis.

24. A BLyS binding protein as defined in any one of clauses 1 to 12 or clause 18, or a pharmaceutical composition as defined in clause 20 for use in therapy.

25. A BLyS binding protein as defined in any one of clauses 1 to 12 or clause 18, or a pharmaceutical composition as defined in clause 20 for use in the treatment of systemic lupus erythematosus, lupus nephritis, systemic sclerosis, cutaneous lupus erythematosus, anti-neutrophil cytoplasmic antibody (ANCA)-associated vasculitis, systemic sclerosis-associated interstitial lung disease, connective tissue disease, and/or connective tissue disease-associated interstitial lung disease.

26. Use of a BLyS binding protein as defined in any one of clauses 1 to 12 or clause 18, or a pharmaceutical composition as defined in clause 20 in the manufacture of a medicament for use in the treatment of disease.

The invention is further illustrated by the following non-limiting examples.

### EXAMPLES

### Example 1—Generation of Antibodies and Lead Panel Analysis

In order to provide the necessary improvement needed for patients it was determined that an antibody with selectivity for BAFF over APRIL and other TNF family receptors, similar or better efficacy to Belimumab, binding to the same epitope as Belimumab, and with an increased affinity and a decreased clearance rate was required to be generated.

In particular, it was hypothesized that an antibody would need a KD of 267 pM (approximately 20-fold better binding than Belimumab, which is already an exceptionally high affinity antibody).

Antibodies were generated and a lead panel of 25 mAbs selected with 38 engineered variants and also developmental risks such as (either or both of) T cell epitope and M54 oxidation risks removed. This provided 90 molecules (67 unique and 23 duplicates) which were then submitted for transfection. Of these, 14 of the unique antibodies and 3 of the duplicates failed to progress into production due to exceptionally low yields.

A number of assays including SPR binding and cell assays, developability screen, and solubility studies and translational screens were carried out to triage the variants.

One of the larger triaging activities (% monomer and thermal stability, high order aggregate, and high molecular weight species heterogeneity and solubility assays) led to the exclusion of a further 20 mAbs for progression and translational profiling further discounted at least 7 mAbs as not suitable for progression. Interestingly, one of the 7 mAbs deemed not suitable for progression due to higher non-specific binding was belimumab. This shows the unpredictable nature of antibody generation given that belimumab is a successfully marketed therapeutic antibody.

Once all assays were completed a lead panel of 9 antibodies was chosen for further analysis.

### Example 2—Genetically Linked Single Chain Antigen Strategy for Hit Screening

The homotrimeric BLyS antigen displays 3 epitopes and as such when generating antibodies to this target avidity effects contribute to the high affinity between the antibody and the antigen.

Due to the limitations of the surface plasmon resonance (SPR) screening platform on an already high affinity antibody, such as belimumab, it is difficult to differentiate individual ultra-high affinity antibodies from a hit panel and so a novel strategy was developed to generate a trimeric soluble BLyS with a reduced number of epitopes for binding. Into a recombinant single chain protein composed of three genetically linked BLyS protomers were introduced a series of mutations targeted to key residues comprising the epitope of Belimumab. Candidate mutations were identified by analysing the BLyS: Belimumab crystal structure and interface alanine scanning. BAFF-neutralizing interaction of belimumab related to its therapeutic efficacy for treating systemic lupus erythematosus. Amino acids Y206/L224/R265 deemed critical in the binding to Belimumab were targeted for substitution for alternatives with different side chain characteristics identified by in silico analysis using OSPREY. The preferred combination generated was Y206W/L224A/R265E. This series of mutations was introduced into 2 protomers which in effect fixed the belimumab epitope in a single chain molecule to one epitope per single chain trimeric unit. Other molecules were constructed with the epitope ablated in all three protomers or with no mutations to act as references for no binding of belimumab or complete binding respectively.

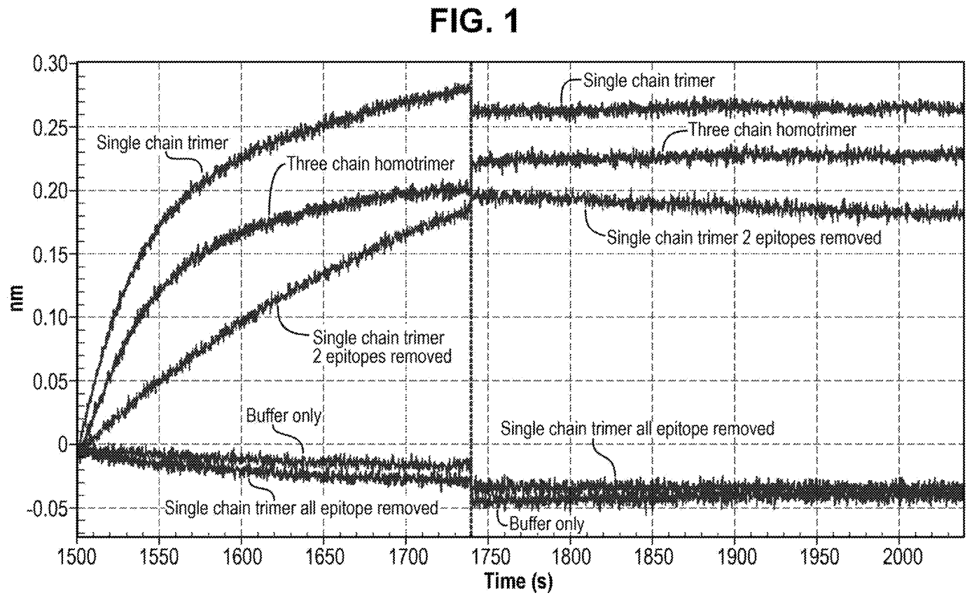

SPR based protein binding assessments (FIG. 1) showed that belimumab binding to the single chain construct with three wild type protomer sequences was similar to an unconstrained (three chain) trimer but binding was ablated if mutations were introduced in all three protomers in the single chain molecule.

Intermediate binding levels were observed for the single chain molecule harbouring one native sequence protomer and two mutated protomers, suggesting that the avidity component to affinity had been removed.

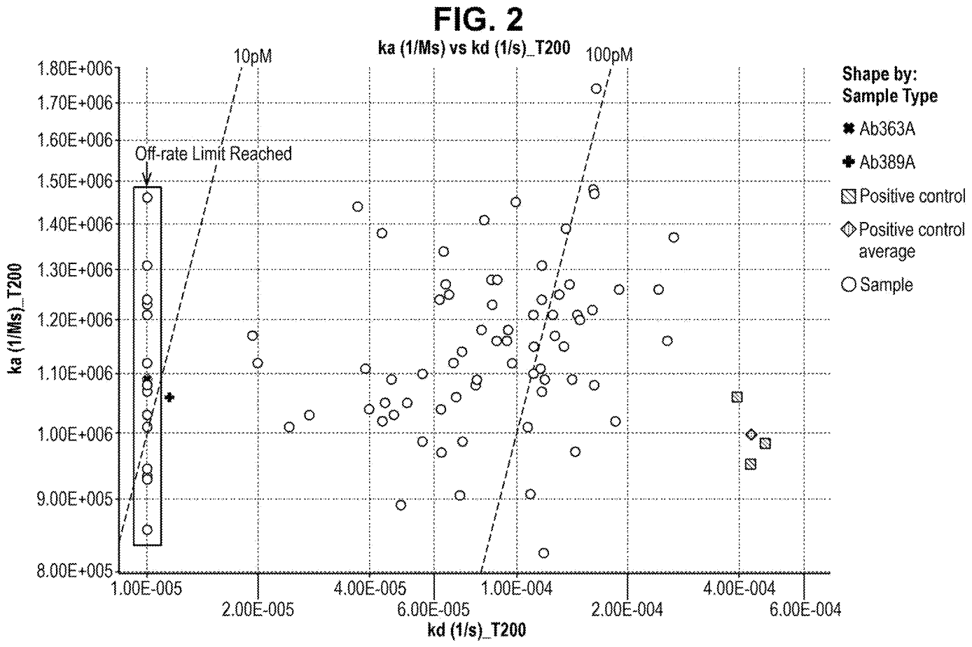

Using the novel antigen, a series of antibodies was screened based upon affinity alone to differentiate those with the highest binding affinity. The screening panel included Belimumab as an internal reference. FIG. 2 shows the range of antibodies screened with those with highest affinity (boxed). Among the high affinity antibodies, two were labeled as Ab363A and Ab389A and selected for progression.

TABLE B
Summary of SEQ ID numbering for Ab363A and Ab389A

| | SEQ ID NOS | | | | | | | | | |
|---|---|---|---|---|---|---|---|---|---|---|
| | HC | | | | | LC | | | | |
| | (HC | | | | | (LC | | | | |
| | DNA) | VH | CDRH1 | CDRH2 | CDRH3 | DNA) | VL | CDRL1 | CDRL2 | CDRL3 |
| Ab363A | 9 | 7 | 1 | 2 | 3 | 10 | 8 | 4 | 5 | 6 |
| | (11) | | | | | (12) | | | | |
| Ab389A | 23 | 21 | 15 | 16 | 17 | 24 | 22 | 18 | 19 | 20 |
| | (13) | | | | | (14) | | | | |

HC: heavy chain; VH: variable heavy chain; CDR: complementarity determining region; LC: light chain; VL: variable light chain.

### Example 3—Binding Affinities to BLyS Using Biacore

Binding of the three antibodies Ab389A, Ab363A, and Belimumab to recombinant human and cynomolgus monkey BLyS was assessed using the capture kinetics method on Biacore T200 (Cytiva) surface plasmon resonance instrument. The antibodies and controls were captured on anti-human Fc capture antibody (Southern Biotech) which was immobilised by primary amine coupling. The assay was run at 37° C. in HBS-EP⁺ buffer and at 25° C. in HBS-EP⁺ buffer at pH 6.0 and pH 7.0. Buffer alone (0 nM analyte concentration) injection was used to double reference the binding curves.

Table 1 shows the affinities of Ab363A, Ab389A, and Belimumab for binding to recombinant human and cynomolgus BLyS.

Ab363A displayed an affinity of 39 pM in comparison to Belimumab which had an affinity of 1.5 nM. This represents a 39-fold improvement in affinity. In comparison, Ab389A had an affinity of 110 pM and represents an affinity improvement of 14-fold in comparison to Belimumab. The affinity of Ab363A for binding to recombinant cynomolgus BLyS was 11 pM and of Ab389A 29 pM, demonstrating cross reactivity.

TABLE 1
Binding of Ab363A, Ab389A, and Belimumab
to Recombinant Human and Cynomolgus BLyS

| | Human BLyS | | Cyno BLyS | |
|---|---|---|---|---|
| | Geometric | | Geometric | |
| | Mean KD (M) | St. Dev | Mean KD (M) | St. Dev |
| Belimumab | 1.51E−09 | 3.67E−10 | 2.78E−10 | 4.73E−11 |
| Ab363A | 3.91E−11 | 5.50E−12 | 1.13E−11 | 3.92E−12 |
| Ab389A | 1.1E−10 | 1.03E−10 | 2.87E−11 | 9.47E−12 |

### Example 4—Binding Affinities of the Antibodies to Recombinant Human and Cynomolgus FcRn Receptors Using Biacore

Ab363A and Ab389A had an approximately 2-fold increase in affinity for the human and cynomolgus FcRn neonatal receptor at pH 6.0 compared to belimumab and an anti-RSV isotype control (Table 2). At pH 7.0 the binding affinity of all the antibodies tested was too weak to obtain steady state affinity values, with the exception of anti-RSV YTE control. This was the case for both human and cyno FcRn. The profile for Ab363A and Ab389A was comparable to the extended anti-RSV control antibody at both pH 7.0 and pH 6.0. Increased affinity for FcRn was provided by “YTE” modifications. Cross reactivity of Ab363A and Ab389A was observed with cynomolgus monkey FcRn.

TABLE 2
Binding of Ab363A, Ab389A, and Belimumab to Recombinant
Human and Cynomolgus FcRn (pH 6.0)

| | human FcRn | cyno FcRn |
|---|---|---|
| Sample | KD (nM) pH 6.0 | KD (nM) pH 6.0 |
| IgG1 Isotype Control | 879 | 749 |
| IgG1 Isotype Control with YTE | 167 | 152 |
| Belimumab | 753 | 760 |
| Ab363A | 428 | 354 |
| Ab389A | 396 | 322 |

### Example 5: Inhibition of B Cell Proliferation

Primary B cells were isolated from human blood ‘cones’, produced as a by-product of the apheresis process and provided by the NHS Blood and Transport Service. ‘Untouched’ CD19+ B cells were isolated via negative selection using an EasySep Direct Human B cell Isolation Kit (StemCell).

2.5 μg/mL anti-IgM F(ab′)2 and the EC80 of recombinant human BLyS trimer (7.9 pM based on the molecular weight of BLyS trimer of 50.7 kDa) or recombinant human BLyS 60mer (1.8 pM based on the molecular weight of BLyS 60mer of 1100 kDa) were prepared at 3× final assay concentration in B cell media. For native BLyS experiments 7.9 pM native human BLyS (present in conditioned media from U937 monocyte cell culture) was used and for recombinant cyno BLyS and EC80 concentration of 26 pM (based on the molecular weight of BLyS trimer of 50.7 kDa) was used. 10 μL added to all wells (except the negative control well—to which 10 μL of 3× final assay concentration (2.5 μg/mL final) anti-IgM F(ab′)2 only added) of 384 well TC treated Alpha plate (Perkin Elmer) using a multidrop (Thermo Fisher). Ab363A, Ab389A, and Belimumab were serially diluted at 3× final assay concentration 1:3 to obtain an 11-point concentration response curve in B cell media before 10 μL added in triplicate to the 384 well TC treated Alpha plate. The anti-RSV YTE was diluted to 3× final assay concentration and 10 μL added to the positive and negative control wells. B cells were counted on a ViCell (Beckman Coulter) and diluted to 1×10⁶ cells/mL. 10 μL B cells (10,000 cells per well) added to the 384 well TC treated Alpha plates using a multidrop. Plates were incubated at 37° C./5% CO₂ for 4 days.

After 4 days CTG (Promega) prepared and 30 μL per well added to the 384 well TC treated Alpha plates using the multidrop. Assay plates incubated for 10 minutes at room temperature before luminescence measured using a Pherastar multimode plate reader (BMG).

Data was transformed into an XY table, with the concentration of Ab363A, Ab3893A, or Belimumab as log 10 M values used for the X axis. For the Y axis, for each donor, the % inhibition values were plotted.

Ab363A, Ab389A, and Belimumab mean pIC50, % Max Inhibition, and slope from six donor B cells for both the BLyS trimer and BLyS 60mer B cell proliferation assay were calculated.

Data is shown in Table 3 from six human B cell donors for inhibition of human BLyS trimer stimulated CD19+ B cell proliferation. Ab363A inhibited proliferation of B cells with mean pIC50=11.02 (IC50=9.5 pM) and for Ab389A the mean pIC50=10.78 (IC50=17 pM). In comparison, the potency of belimumab was mean pIC50=9.64 (IC50=230 pM). This demonstrates that Ab363A is a potent neutralizer of human BLyS-stimulated human B cell proliferation, with an approximate 24-fold improvement in potency compared to belimumab. For Ab389A, the fold increase in potency compared to belimumab was approximately 14-fold. All the antibodies tested were fully efficacious at inhibiting the proliferation response.

TABLE 3
Inhibition of human BLyS trimer-induced human CD19+
primary B cell proliferation by Ab363A, Ab389A,
and Belimumab (N = 6 donors)

| | Human BLyS Trimer | | | | | |
|---|---|---|---|---|---|---|
| | | | Maximum % | | | |
| | pIC50 Mean | | Inhibition | | Slope | |
| | | ±95% | | ±95% | | ±95% |
| | Mean | CI | Mean | CI | Mean | CI |
| Ab363A | 11.02 | 10.9-11.1 | 110.98 | 94.6-127.4 | 2.91 | 1.7-4.1 |
| Ab389A | 10.78 | 10.6-10.9 | 126.38 | 112.5-140.3 | 1.63 | 1.1-2.2 |
| Belimumab | 9.64 | 9.4-9.8 | 146.08 | 124.0-168.1 | 0.87 | 0.7-1.0 |

Data is shown in Table 4 for inhibition of cyno BlyS trimer stimulated CD19+ B cell proliferation. Ab363A and Ab389A completely neutralized cyno BlyS trimer protein in the B cell proliferation assay, with mean pIC50=10.51 (IC50=31 pM) and was approximately 10× more potent than belimumab. For Ab389A mean pIC50=10.46 (IC50=34 pM).

TABLE 4
Inhibition of cyno BlyS trimer-induced human CD19+ primary B cell
proliferation by Ab363A, Ab389A, and Belimumab (N = 6 donors)

| | Cyno BLyS trimer | | | | | | | | |
|---|---|---|---|---|---|---|---|---|---|
| | | | | Maximum % | | | | | |
| | pIC50 | | | Inhibition | | | Slope | | |
| | | ±95% CI | | | ±95% CI | | | ±95% CI | |
| | | lower | Upper | | lower | Upper | | lower | Upper |
| | Mean | CI | CI | Mean | CI | CI | Mean | CI | CI |
| Ab363A | 10.51 | 10.43 | 10.59 | 107.37 | 93.76 | 120.97 | 2.79 | 2.32 | 3.27 |
| Ab389A | 10.46 | 10.35 | 10.56 | 106.96 | 89.07 | 124.86 | 2.82 | 1.48 | 4.15 |
| Belimumab | 9.46 | 9.08 | 9.83 | 106.64 | 92.03 | 121.24 | 1.58 | 0.98 | 2.18 |

Table 5 shows data for inhibition of human BLyS 60mer stimulated B cell proliferation. Ab363A and Ab389A inhibited human BLyS 60mer-stimulated proliferation of B cells with a mean pIC50=10.13 (IC50=74.1 pM) and mean pIC50=9.64 (IC50=229 pM) respectively. In comparison, Belimumab showed a mean pIC50=5.86 (IC50=1.4 μM). This demonstrates that Ab363A is a potent neutralizer of human BLyS 60mer-stimulated B cell proliferation with an approximately 18,000-fold improvement in potency (possibly due to increased affinity) compared to the parent molecule belimumab. No effect of anti-RSV-YTE IgG isotype control was noted and both antibodies were fully efficacious at inhibiting the proliferation response.

TABLE 5
Inhibition of Human BLyS 60mer-induced human CD19+ B cell
proliferation by Ab363A, Ab389A and Belimumab (N = 6 donors)

| | BLyS 60mer | | | | | |
|---|---|---|---|---|---|---|
| | pIC50 | | Maximum % | | | |
| | Mean | | Inhibition | | Slope | |
| | | ±95% | | ±95% | | ±95% |
| | Mean | CI | Mean | CI | Mean | CI |
| Ab363A | 10.13 | 9.9-10.4 | 123.33 | 98.2-148.5 | 1.04 | 0.8-1.2 |
| Ab389A | 9.64 | 9.3-10 | 119.41 | 79.6-159.2 | 1.15 | 0.5-1.8 |
| Belimumab | 5.86 | 5.3-6.4 | 154.27 | 105.8-202.7 | 1.89 | 0.8-3.0 |

Ab363A binds to the same epitope as belimumab which is only partially exposed in BLyS 60-mer and includes the FLAP region of BLyS which mediates stabilization of BLyS 60-mer. Belimumab and Ab363A do not bind preformed 60-mer as assessed by SPR, consistent with the epitope being only partially exposed. Both antibodies bind the FLAP region in soluble 3-mer and have potential to block new formation of 60-mer (demonstrated previously for belimumab using gel filtration experiments).

Although the translational consequence of inhibition of 60-mer activity is difficult to determine, published in vitro cell experiments indicate that 60-mer may be more effective at signalling than the trimer, due to higher avidity affects; particularly via the TACI receptor, which is implicated in T-independent activation of autoreactive B cells in the tissues. Thus, blocking 60-mer may be an upside to the activity of Ab363A in autoimmunity and will be explored further.

Table 6 shows inhibition of native BLyS stimulated CD19+ B Cell proliferation. Ab363A and Ab389A inhibited native human BLyS stimulated proliferation of CD19+ human B cells with mean pIC50=11.35 (IC50=4.5 pM) and mean pIC50=11.1 (IC50=8 pM), respectively. Ab363A was 26× more potent than belimumab which showed mean pIC50=9.93 (IC50=117 pM). Both antibodies had comparable maximum effects, with a range of 70-100% depending on the donor. These results provide evidence that Ab363A and Ab389A are active against native BLyS with potency in line with the recombinant BLyS assays.

TABLE 6
Inhibition of native human BLyS-induced human CD19+ primary B cell proliferation
by Ab363A and Belimumab (N = 7 donors) and Ab389A (N = 4 donors)

| | Native human BLyS | | | | | | | | |
|---|---|---|---|---|---|---|---|---|---|
| | | | | Maximum % | | | | | |
| | pIC50 | | | Inhibition | | | Slope | | |
| | | ±95% CI | | | ±95% CI | | | ±95% CI | |
| | | Lower | Upper | | Lower | Upper | | Lower | Upper |
| | Mean | CI | CI | Mean | CI | CI | Mean | CI | CI |
| Ab363A | 11.35 | 11.22 | 11.48 | 87.54 | 70.68 | 104.4 | 1.86 | 1.43 | 2.3 |
| Ab389A | 11.10 | 10.96 | 11.24 | 77.38 | 68.33 | 86.43 | 2.04 | 0.97 | 3.11 |
| Belimumab | 9.93 | 9.76 | 10.09 | 88.65 | 75.18 | 102.13 | 1.75 | 0.79 | 2.7 |

### Example 6: Blocking of BLyS-Receptor Binding by Ab363A, Ab389A, and Belimumab

BLyS labelled with europium competes with antibodies for inhibition of BLyS binding to wild type BAFF receptors (BAFFR) stably overexpressed in CHO K1 cells.

Eleven point 1:2 serial dilutions of Ab363A, Ab389A (starting from 0.15 nM final concentration) or Belimumab (starting from 5 nM final concentration) were prepared and 10 μL volumes transferred to test assay plates. An equal volume of BLYS labelled with europium was added to every well, to give a final concentration of 15 pM (based on the molecular weight of BLyS trimer). The contents of the plates were mixed prior to addition of 3×10⁴ CHO K1 BAFFR cells per well (in a 20 μL volume of cell medium). The plates were pulse centrifuged and shaken briefly to mix the contents before incubating at 37° C./5% CO₂ for 60-75 minutes. Following incubation, the plates were centrifuged, and medium was aspirated. The cells were washed twice to remove any unbound antibody. A 30 μL volume of room temperature DELFIA Enhancement solution (Perkin Elmer) was dispensed into every well and sealed plates were placed on a shaker for 15 minutes.

Antibodies were tested in triplicate or duplicate samples to generate pIC50 values from three independent experiments. 95% confidence intervals with lower and upper limits were calculated for pIC50, slopes, and percent maximum efficacy of inhibition at the highest concentration tested.

Further experiments were performed to assess inhibition of BLyS binding to BAFFR, TACI and BCMA receptors. Human BAFFR: Fc, TACI: Fc, and BCMA: Fc receptors (1 μg/mL) were coated on standard bind MSD plates overnight at 4° C. Plates were washed (×3) in phosphate-buffered saline (PBS) 0.1% Tween 80 and blocked with 1% bovine serum albumin (BSA) at room temperature for 1 hour. Ab363A, Ab389A, or Belimumab were serial diluted 1:2 to obtain 11-point concentration response curves in PBS plus 0.1% BSA at twice the final assay concentration. The highest concentration tested was either 500 pM or 1 nM. Recombinant human BLyS was diluted to twice final assay concentration (15 or 30 pM based on the molecular weight of BLyS trimer) and equal volumes mixed with Ab363A, Ab389A, or Belimumab dilutions to generate multiple curves from three independent experiments. Plates were shaken (600 RPM) for 1 hour at room temperature to allow BLyS binding to antibody. Positive control columns contained BLyS (15 or 30 pM final) plus anti RSV YTE isotype control (500 pM or 1 nM) and negative controls contained PBS 0.1% BSA with isotype control. After 1 hour the mixture was added to washed MSD plates coated with separate BLyS receptors and the plates incubated at room temperature for 2 hours while shaking. Following wash steps, the amount of bound BLyS was detected by anti-BLyS biotinylated-streptavidin sulfotag detection antibodies and the luminescence quantified using Meso Scale Discovery (MSD) Sector Instrument.

The ActivityBase software generates a curve fit and a pIC50 value (see Tables 7, 8, and 9) for each curve. All curves were checked and when poor fits were observed the curves were rejected. No more than 3 points per curve were excluded to obtain a curve fit, including the points that can be automatically excluded by the software. 95% confidence intervals with lower and upper limits were calculated for pIC50, slopes, and percent maximum inhibition at the highest concentration tested.

### Example 7: Inhibition of Human BLyS Trimer Binding to BAFF-R, TACI, and BCMA Receptors

Tables 7-9 show inhibition of BLyS binding to BAFFR: Fc, BCMA: Fc, and TACI: Fc receptors with Ab363A, Ab389A, and Belimumab, respectively.

Ab363A inhibited binding of human BLyS trimer to BAFF-R: Fc, BCMA: Fc, and TACI: Fc receptors with Mean pIC50=10.9, 10.9 (IC50 13 pM), and 10.7 (IC50=20 pM), respectively.

Ab389A inhibited binding to these receptors with mean pIC50=10.8 (IC50=16 pM), pIC50=10.8 (IC50=16 pM), and 10.7 (IC50 20 pM), respectively.

In comparison, Belimumab inhibited binding to all three receptors but with an overall 10-fold reduced potency (Table 9). Ab363A and Ab389A were fully efficacious at inhibiting the binding of BLyS to its receptors with no effect of the anti-RSV IgG YTE isotype control (data not shown).

TABLE 7
Inhibition of human BLyS binding to BAFFR,
TACI, and BCMA receptors by Ab363A
Ab363A

| | | | Maximum % | | | |
|---|---|---|---|---|---|---|
| | pIC50 Mean | | Inhibition | | Slope | |
| | Mean | ±95% CI | Mean | ±95% CI | Mean | ±95% CI |
| BAFFR | 10.9 | 9.9-11.8 | 101 | 97-106 | 2.3 | 1.4-3 |
| BCMA | 10.9 | 10.1-11.7 | 100 | 98-101 | 1.9 | 1.2-2.6 |
| TACI | 10.7 | 10.3-11 | 101 | 97-103 | 2.7 | 1.4-4 |

TABLE 8
Inhibition of human BLyS binding to BAFFR,
TACI, and BCMA receptors by Ab389A
Ab389A

| | | | Maximum % | | | |
|---|---|---|---|---|---|---|
| | pIC50 Mean | | Inhibition | | Slope | |
| | Mean | ±95% CI | Mean | ±95% CI | Mean | ±95% CI |
| BAFFR | 10.8 | 9.4-12.1 | 100 | 97-103 | 2.2 | 1.4-2.9 |
| BCMA | 10.8 | 9.5-12 | 100 | 99-101 | 2.3 | 1.2-3.4 |
| TACI | 10.7 | 10-11.3 | 101 | 97-104 | 2.5 | 1.6-3.4 |

TABLE 9
Inhibition of human BLyS binding to BAFFR,
TACI, and BCMA receptors by belimumab
Belimumab

| | | | Maximum % | | | |
|---|---|---|---|---|---|---|
| | pIC50 Mean | | Inhibition | | Slope | |
| | Mean | ±95% CI | Mean | ±95% CI | Mean | ±95% CI |
| BAFFR | 10.0 | 9.7-10.3 | 103 | 84-121 | 1.3 | 1-1.5 |
| BCMA | 9.9 | 9.9-9.9 | 99 | 97-100 | 1.4 | 0.7-2.1 |
| TACI | 9.7 | 9.2-10.2 | 91 | 79-104 | 1.3 | 1-1.6 |

In addition, human BLyS trimer binding to BAFFR over-expressed on CHO-K1 cells was also inhibited with a mean pIC50=11.05 (IC50=9 pM) by Ab363A and a mean pIC50=10.9 (IC50=13 pM) for Ab389A.

TABLE 10
Inhibition of BLyS binding to BAFFR over-expressed in CHO-K1 cells

| | Mean pIC50 | | Maximum % Inhibition | | Slope | |
|---|---|---|---|---|---|---|
| | | 95% CI | | 95% CI | | 95% CI |
| | Mean | Range | Mean | Range | Mean | Range |
| Ab363A | 11.05 | 10.89-11.22 | 102.2 | 101.7-102.6 | 1.76 | 0.41-3.11 |
| Ab389A | 10.9 | 10.2-11.6 | 100.7 | 84.8-116 | 1.2 | 0.002-2.5 |
| Belimumab | 10.52 | 10.42-10.62 | 96.1 | 94.93-97.29 | 1.06 | 0.66-1.46 |

### Example 8—Crystal Structure of FAb Fragment of mAb Ab363A in Complex with Human BLyS (hBLyS)

The Fab fragment of Ab363A was generated by digestion using a Genovis FabALACTICA Fab kit according to the manufacturer's instructions. The Fab fragment was subsequently separated from Fc and other components by Protein A magnetic bead affinity purification (Cytiva). Purified Fab and hBLyS were mixed at 1.3 (Fab):1 (hBLyS) molar ratio and incubated at room temperature for 1-2 hours. Size exclusion chromatography using 50 mM HEPES pH6.8, 150 mM NaCl buffer was used to isolate the Ab363A Fab: BLyS antigen complex which was then concentrated to 2.57 mg/mL.

The complex was crystallised for structure determination by X-ray crystallography. Sitting drop vapour diffusion was carried out at 20° C. by combining the purified protein complex with well buffer (22% PEG1000, 0.1M trisodium citrate pH5.5) at 1 (protein): 2 (well buffer) drop ratio. Crystals obtained were briefly cryoprotected in buffer (90% well buffer+10% glycerol) after 12 days growth, and flash-cooled in liquid nitrogen.

X-ray diffraction data were collected on beamline I03 (Diamond Light Source) from a crystal at 100K. The data were processed using Global Phasing Autoproc STARANISO (with XDS) to 2.23 Å resolution and indexed as a trigonal crystal system with space group P3121 and cell dimensions a=b=120.03 Å, c=326.95 Å, α=β=90°, γ=120°. (See Figures).

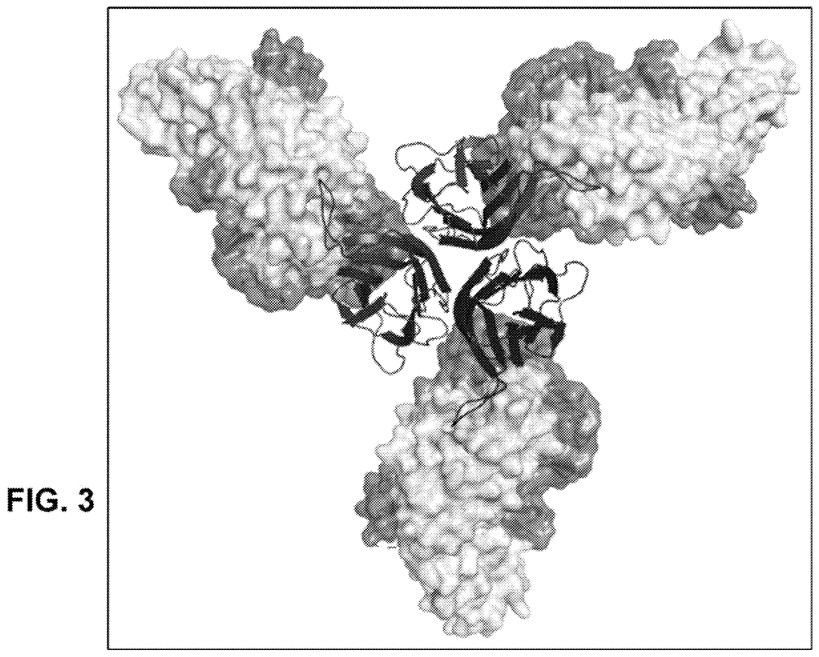

FIG. 3 shows the crystal structure of a BLyS trimer (displayed in black ribbon representation) bound to three Ab363A Fab fragments (displayed in grey (heavy chain) and light grey (light chain) surface representation) at 2.23 Å resolution. The Fabs are bound at equivalent epitopes around the BLyS trimer. An average interface area of 1,025 Å² and buried surface area of 2319 Å² for each Fab with the BLyS trimer were determined using PISA and PyMOL respectively. Considering the amount of interface surface area available to confer binding, the affinity of Ab363A is exceptionally high and exceeds the binding efficiencies of all comparable protein-protein interactions in two benchmark studies.

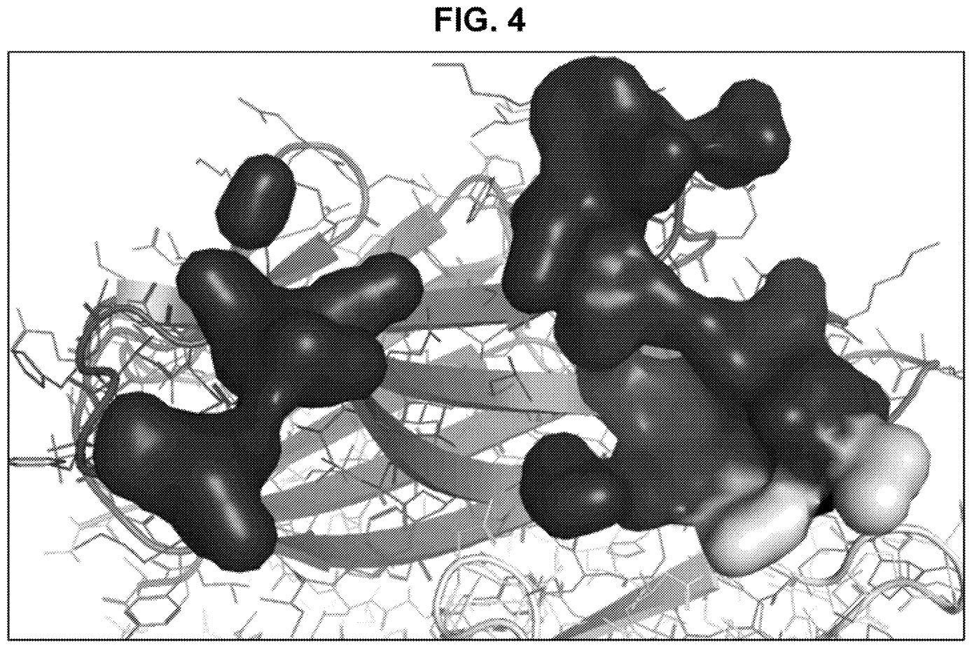

FIG. 4 shows the BLyS trimer epitope (displayed in surface representation) interacting with an individual Ab363A Fab, within a contact radius of 4.5 Å using PYMOL. The epitope is formed from two BLyS protomers of the trimer: a principal BLyS protomer providing most of the epitope (shown in black) and comprising K¹⁶⁰G¹⁶¹S¹⁶²Y¹⁶³T₂₀₅Y²⁰⁶A²⁰⁷M²⁰⁸G²⁰⁹K²¹⁵G²²¹D²²²L²²⁴S²²⁵L²²⁶R²³¹I²³³P²⁶⁴R²⁶⁵E²⁶⁶, while an adjacent BLyS protomer of the trimer extends the contact via additional epitope L²⁴⁰N²⁴² (shown in light grey).

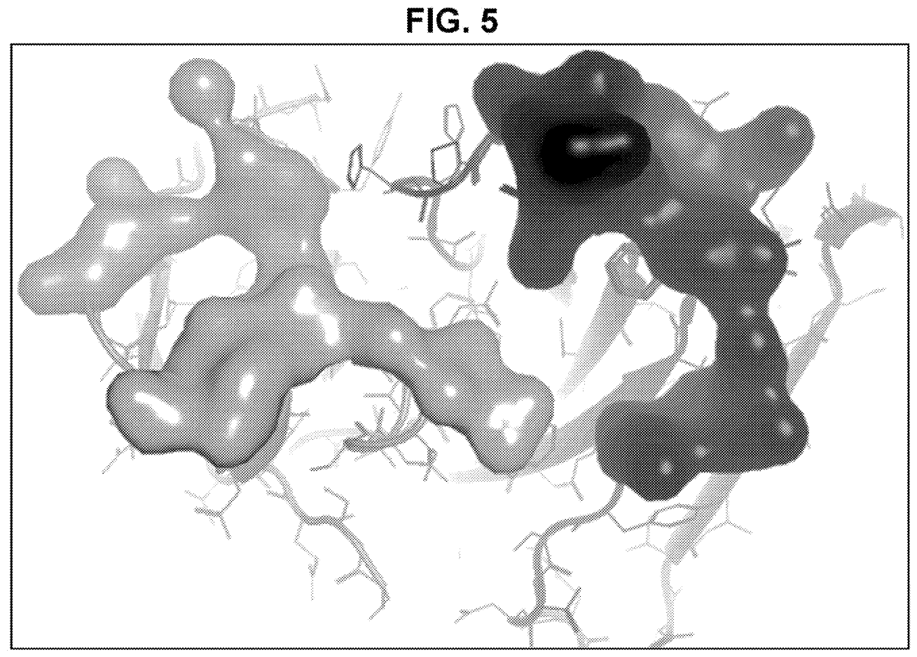

FIG. 5 shows the Ab363A Fab paratope (displayed in surface representation) interacting with the BLyS trimer within a contact radius of 4.5 Å using PYMOL. The Fab heavy chain contributes paratope N³¹M⁵⁴F⁵⁵G⁵⁶T⁵⁷K⁵⁹D¹⁰¹P¹⁰²L¹⁰³L¹⁰⁴ (shown in black) and the Fab light chain contributes paratope L²⁷R²⁸Y²⁹Y³⁰Y³¹K⁵⁰S⁶⁴S⁶⁵S⁶⁶S⁹³G⁹⁴N⁹⁵ (shown in light grey).

For belimumab, those residues that interact with BlyS within a contact radius of 4.5 Å (using PYMOL) are: Fab heavy chain residues N³¹M⁵⁴F⁵⁵G⁵⁶T⁵⁷K⁵⁹D¹⁰¹L¹⁰²L¹⁰³L¹⁰⁴P¹⁰⁶; and Fab light chain residues L²⁷R²⁸S²⁹Y³⁰Y³¹K⁵⁰S⁶⁵S⁶⁶G⁶⁷S⁹³G⁹⁴N⁹⁵. The belimumab Fab/BLYS complex is obtained from PDB entry 5y9j. A comparison with the belimumab Fab/BlyS crystal structure suggests that it is those residues within the Ab363A CDRs that result in the improved affinity of Ab363A for BLyS.

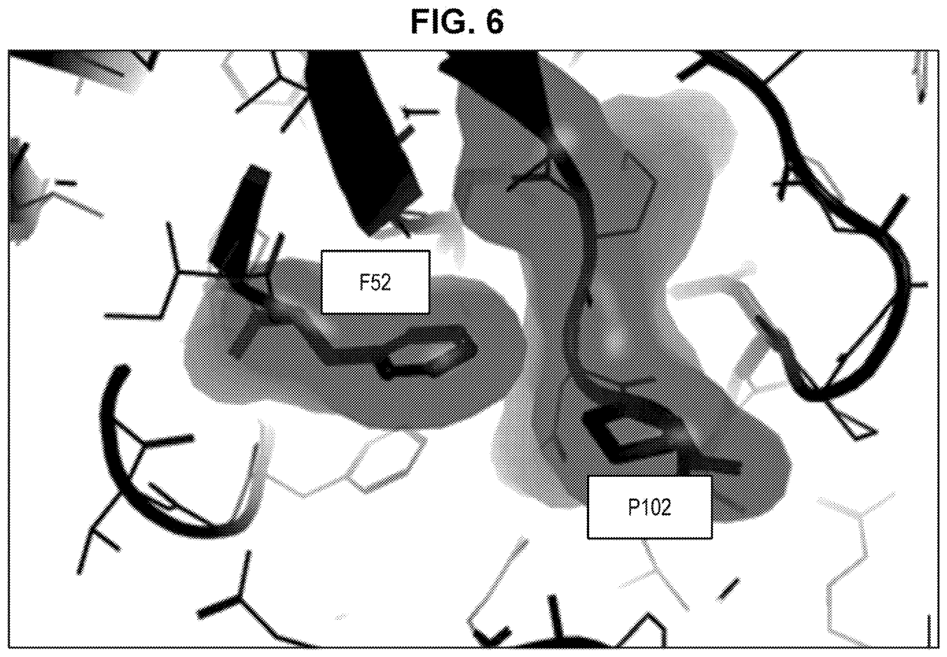

FIG. 6 shows surface representation (using PYMOL) of CDR_H2 F52 highlighting its space filling and pi-stacking properties and proximity to CDRH3 P102 in the Ab363A Fab paratope.

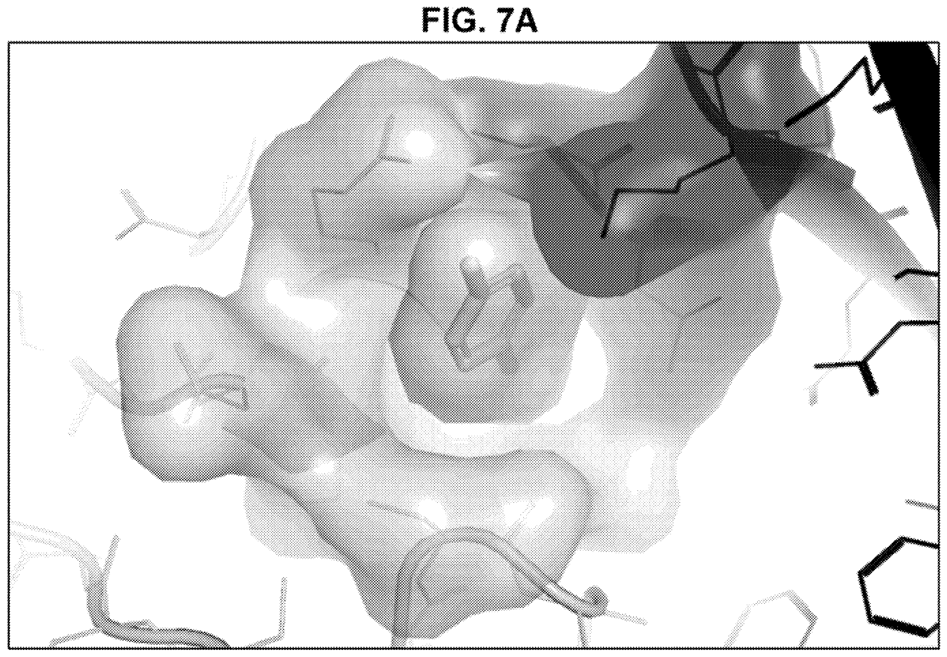

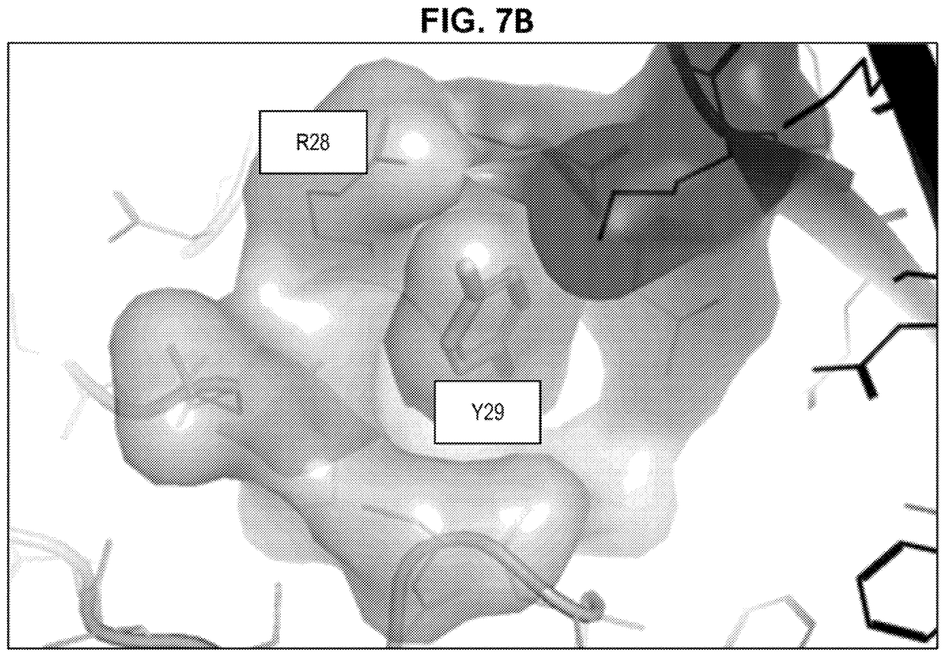

FIG. 7A shows CDRL1 Y29 sidechain space filling the pocket where it resides and additionally hydrogen-bonding to the CDRL1 R28 in the Ab363A Fab paratope (displayed in surface representation using PYMOL). FIG. 7B provides labeling of the residues shown in FIG. 7A.

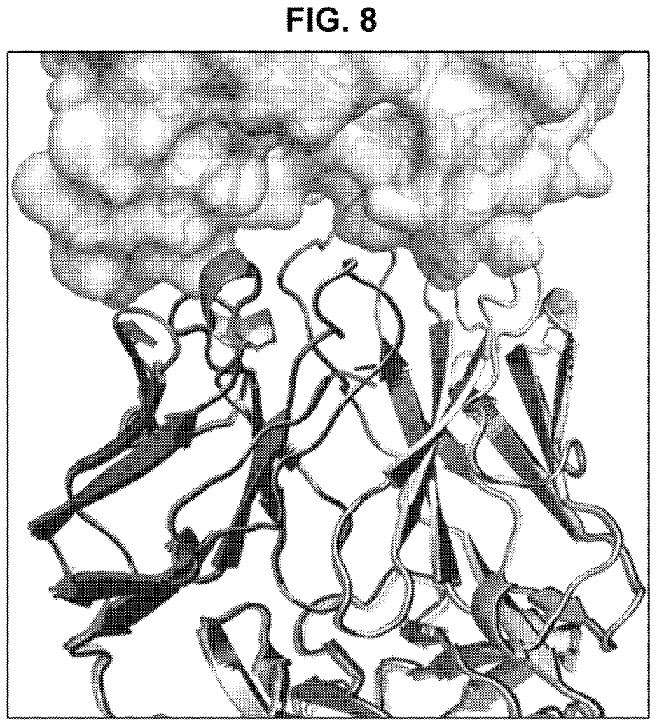

FIG. 8 shows an overlay of the three Ab363A Fab/BLyS protomer subunits of the Ab363A Fab/BLyS trimer structure. The subunits are coloured black, grey, and light grey and the overlay highlights the close structural similarity of the three Ab363A Fab/BLyS contact interfaces in the trimer. BLyS is shown with a representative molecular surface from one of the overlaid BLyS protomers.

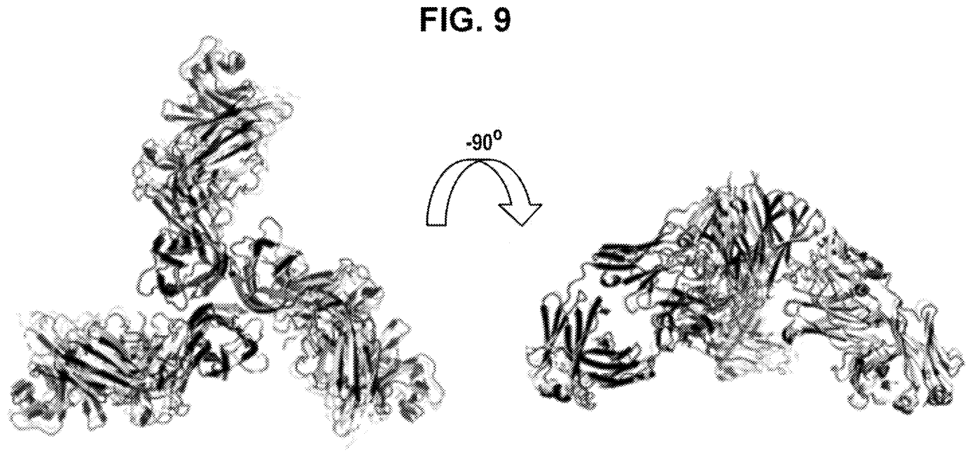

FIG. 9 shows an overlay of Ab363A Fab/BLyS (black) and belimumab Fab/BLyS (light grey) crystal structures. It highlights the comparable 3-fold symmetry, interaction geometry and stoichiometry. The belimumab Fab/BLyS complex is obtained from PDB entry 5y9j.

### Example 9—Epitope Comparison of Ab363A with Belimumab

Structural experiments demonstrated that Ab363 binds to a comparable epitope on BLyS to belimumab. In order to demonstrate that Ab363A binds to a comparable epitope on BLyS as belimumab, Fab fragments enzymatically generated from anti-BLyS mAb Ab363A were mixed with BLyS trimer and the resulting complex was crystallized by sitting drop vapour diffusion. X-ray diffraction data obtained from a resulting crystal was used to solve a 2.23 Å resolution crystal structure by Molecular Replacement employing starting models derived from a published belimumab Fab/BLyS complex. The crystal structure shows the BLyS trimer in complex with three Fab fragments consistent with a stoichiometry of one Fab to one BLyS protomer. This stoichiometry was also observed with the belimumab: BLyS structure, except in this case the trimeric unit is formed by crystal symmetry.

Superimposing the three BLyS: Fab subunits of the trimeric complex shows that the interfaces are comparable and they bind in the same manner despite structure refinement in which non-crystallographic symmetry constraints were strictly avoided. Furthermore, the orientation of the Ab363A Fabs relative to the BLyS trimer is reminiscent of that observed for belimumab.

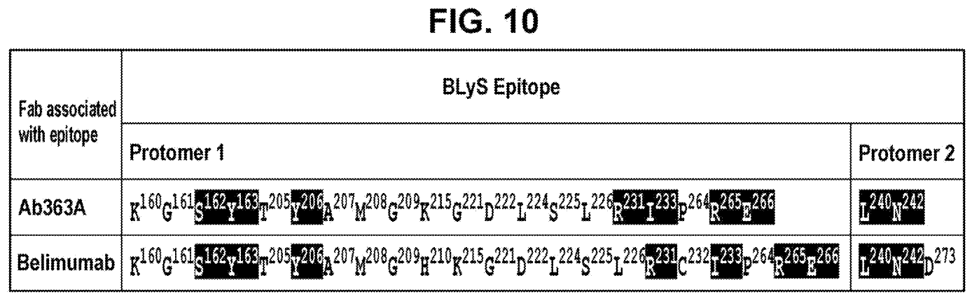

As previously shown by Shin and colleagues (Shin et al.), the epitope on BLyS for belimumab covers three regions: a receptor binding site, a flap region implicated in 60mer formation, and a small region on a neighbouring protomer of the trimer. There are small differences in shape complementarity and interface area for Ab363A and belimumab Fabs with BLyS, most likely due to the mutational differences residing in the interface area. The total interface area for Ab363A Fab with BLyS trimer is approximately 1025 Å² compared to a slightly smaller area of 985 Å² for the belimumab Fab-BLyS interface. The configuration and epitope of Ab363A Fab relative to BLyS appear to be essentially identical to that observed for belimumab Fab with BLyS. (FIG. 10)

### Example 10—PK/PD Determination in Cynomolgus Monkeys

A study was conducted to investigate the intravenous pharmacokinetic (PK) and pharmacodynamic (PD) parameters of Ab363A in the male cynomolgus monkey. A total of twelve animals divided into three groups were used. Group 1 received Ab363A at 0.5 mg/kg and were sampled up to Day 379. Group 2 received Ab363A at 2.0 mg/kg and were sampled up to Day 371. Group 3 received vehicle only and were sampled until Day 84. Throughout the study, samples were collected to measure drug levels for PK, total BLyS levels for target engagement (TE), B cell counts for pharmacodynamic (PD) effects, and LC-MS analysis to test for any chemical modifications to the antibody from in vivo processes. Additional samples were also taken for anti-drug antibody (ADA) analysis.

Ab363A had very low clearance and low Vss, with clearance values that were approximately 1/10 and Vss values approximately ⅓ of the values reported for belimumab.

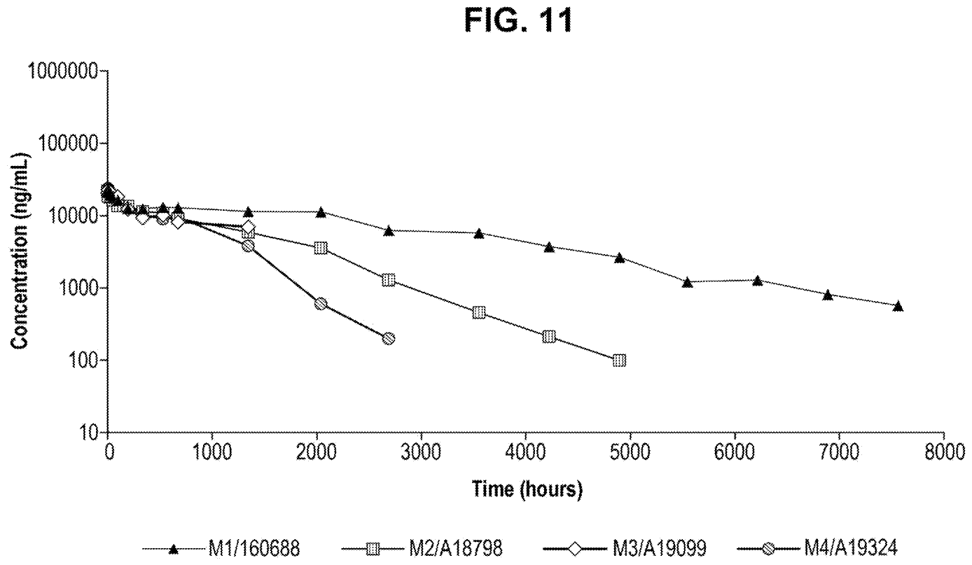

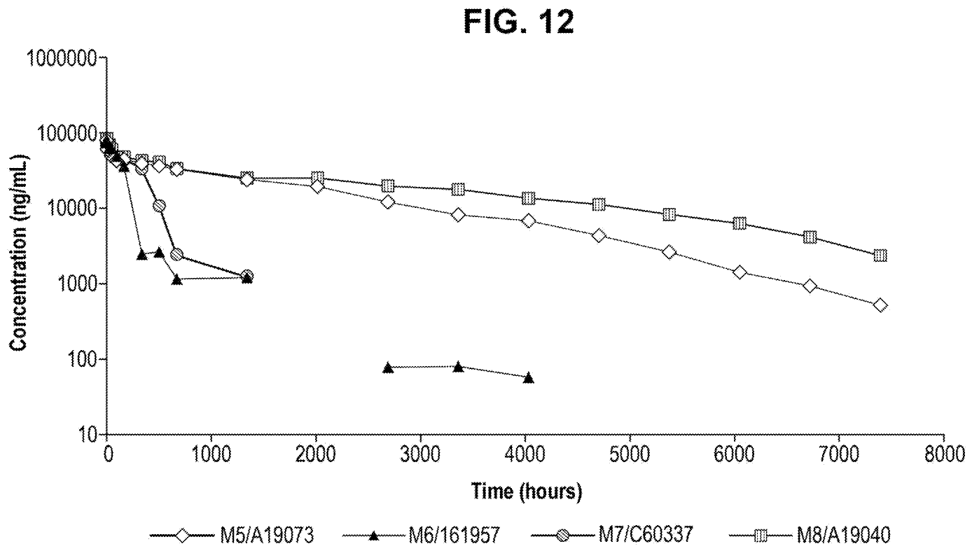

The pharmacokinetic parameters derived from analyses of the experimental data are presented in Table 11. The Ab363 concentration-time data are shown graphically in FIG. 11 and FIG. 12, respectively.

TABLE 11
Pharmacokinetic Parameters of Ab363A in Cynomolgus Monkey
IV Pharmacokinetic Parameters - Group 1 (0.5 mg/kg)

| | Monkey 1 | | | Monkey 4 ^{a} | | |
|---|---|---|---|---|---|---|
| | 160688 (non- | Monkey 2 | Monkey 3 ^{a} | A19324 (non- | | |
| | naïve, pre- | A18798 | A19099 | naïve, pre- | | |
| | dose ADA-) | (naïve) | (naïve) | dose ADA-) | Mean ± SD | |
| Body weight (kg) | 8.20 | 8.95 | 9.15 | 9.70 | 9.00 ± 0.62 | |
| Dose (mg/kg) | 0.499 | 0.495 | 0.501 | 0.500 | 0.499 ± 0.003 | |
| Cₘₐₓ (μg/mL) | 23.9 | 21.8 | 23.3 | 23.7 | 22.9 | (n = 2) |
| Tₘₐₓ (h) | 3.08 | 0.333 | 3.10 | 0.283 | 1.71 | (n = 2) |
| AUC_{0-inf} (h*μg/mL) | 45500 | 19000 | NR | 13600 | 32300 | (n = 2) |
| CL (mL/day/kg) | 0.264 | 0.626 | NR | 0.881 | 0.445 | (n = 2) |
| Vₛₛ (mL/kg) | 25.1 | 27.4 | NR | 24.6 | 26.3 | (n = 2) |
| Half-life (h) | 1300 | 562 | NR | 347 | 931 | (n = 2) |
| MRT | 2290 | 1050 | NR | 669 | 1670 | (n = 2) |

IV Pharmacokinetic Parameters - Group 2 (2 mg/kg)

| | | Monkey 6 ^{a} | Monkey 7 ^{a} | | | |
|---|---|---|---|---|---|---|
| | Monkey 5 | 161957 (non- | C60337 (non- | Monkey 8 | | |
| | A19073 | naïve, pre- | naïve, pre- | A19040 | | |
| | (naïve) | dose ADA-) | dose ADA-) | (naïve) | Mean ± SD | |
| Body weight (kg) | 8.15 | 7.65 | 8.65 | 9.65 | 8.53 ± 0.85 | |
| Dose (mg/kg) | 2.00 | 2.01 | 2.00 | 2.02 | 2.01 ± 0.01 | |
| Cₘₐₓ (μg/mL) | 79.6 | 81.6 | 72.1 | 84.3 | 82.0 | (n = 2) |
| Tₘₐₓ (h) | 0.267 | 0.250 | 3.03 | 0.250 | 0.259 | (n = 2) |
| AUC_{0-inf} (h*μg/mL) | 92000 | 13200 | 20600 | 135000 | 114000 | (n = 2) |
| CL (mL/day/kg) | 0.521 | 3.66 | 2.32 | 0.360 | 0.441 | (n = 2) |
| Vₛₛ (mL/kg) | 38.6 | 43.1 | 26.8 | 38.4 | 38.5 | (n = 2) |
| Half-life (h) | 900 | 622 | 215 | 1220 | 1060 | (n = 2) |
| MRT | 1780 | 283 | 277 | 2560 | 2170 | (n = 2) |

^{a} Indicates that the animals tested positive for ADA. PK parameter values from ADA positive animals were excluded from the mean calculations.
NR—not reportable due to AUC extrapolation >20%

The apparent elimination half-life was approximately 40 days, on average. Ab363A pharmacokinetics were similar at the two dose levels tested (0.5 and 2 mg/kg). Two of the four monkeys in each dose cohort tested positive for anti-drug antibodies during the study. In the vehicle group, all monkeys tested negative for anti-drug antibodies at all time points tested. Three of the four drug-treated animals that tested positive for ADA were previously exposed to an unrelated therapeutic monoclonal antibody. Clearance was generally higher and half-life values were generally lower in animals that tested positive for ADA.

Total BLyS concentrations were evaluated as a measure of target engagement. Monkeys given Ab363A had increased total BLyS concentrations after dose administration, consistent with target engagement, whereas no consistent changes were observed in the vehicle group. Sharp decreases in total BLyS were observed in animals which were ADA positive, consistent with accelerated mAb clearance and loss of target engagement in ADA positive animals as a result of either a binding site-specific ADA or increased clearance of the ADA bound complex.

Intact antibody mass was confirmed in most samples obtained from Group 2 animals, and no clipping or other modifications were observed by LC-MS. Variant peaks were observed at +162 Da (+Hexose) and increased slightly over time. The +Hexose modification is a common variant, and levels of changes observed for Ab363A would not be expected to have any impact. At higher dose levels, specific sites may need to be assessed for +Hex levels and any subsequent impact on target binding.

In monkeys given 2 mg/kg, there was a trend toward decreasing frequency and absolute numbers of total CD20+ B cells seen by Day 84 (week 12) for all animals, regardless of ADA status.

Two animals in the 2 mg/kg group and 2 monkeys in the 0.5 mg/kg group which were confirmed to be ADA positive recovered their total CD20+ B cell frequency between Weeks 16 and 29. Decreases in CD20+ B cells persisted in the remaining 2 monkeys given 2 mg/kg and one animal given 0.5 mg/kg while antibody levels and total BLys levels were still elevated compared to baseline.

SEQUENCE LISTING

SEQ ID NO: 1: Ab363A CDRH1
NDAIN
SEQ ID NO: 2: Ab363A CDRH2
GIFPMFGTAKYSQNFQG
SEQ ID NO: 3: Ab363A CDRH3
SRDPLLYPHHALSP
SEQ ID NO: 4: Ab363A CDRL1
QGDSLRYYYAS
SEQ ID NO: 5: Ab363A CDRL2
GKNNRPS
SEQ ID NO: 6: Ab363A CDRL3
SSRDSSGNNWV
SEQ ID NO: 7: Ab363A VH
QVQLQQSGAEVKKPGSSVRVSCKASGGTFNNDAINWVRQAPGQGLEWMGGIFPMFGTAKYSQNFQGRVAITADEST
GTASMELSSLRSEDTAVYYCARSRDPLLYPHHALSPWGRGTMVTVSS
SEQ ID NO: 8: Ab363A VL
SSELTQDPAVSVALGQTVRVTCQGDSLRYYYASWYQQKPGQAPVLVIYGKNNRPSGIPDRFSGSSSGNTASLTITGAQ
AEDEADYYCSSRDSSGNNWVFGGGTELTVL
SEQ ID NO: 9: Ab363A Heavy chain with YTE
QVQLQQSGAEVKKPGSSVRVSCKASGGTFNNDAINWVRQAPGQGLEWMGGIFPMFGTAKY
SQNFQGRVAITADESTGTASMELSSLRSEDTAVYYCARSRDPLLYPHHALSPWGRGTMVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEL
LGGPSVFLFPPKPKDTLYITREPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO: 10: Ab363A Light chain
SSELTQDPAVSVALGQTVRVTCQGDSLRYYYASWYQQKPGQAPVLVIYGKNNRPSGIPDR
FSGSSSGNTASLTITGAQAEDEADYYCSSRDSSGNNWVFGGGTELTVLGQPKAAPSVTLF
PPSSEELQANKATLVCLISDFYPGAVTVAWKADSSPVKAGVETTTPSKQSNNKYAASSYL
SLTPEQWKSHRSYSCQVTHEGSTVEKTVAPTECS
SEQ ID NO: 11: Ab363A Nucleotide sequence of Heavy chain
CAAGTACAGTTGCAGCAGTCCGGAGCGGAAGTCAAGAAGCCGGGCTCCAGCGTGCGCGTGTCCTGTAAAGCCAG
CGGCGGCACCTTCAACAACGATGCCATTAACTGGGTCCGCCAGGCCCCGGGACAGGGTCTTGAATGGATGGGCG
GGATCTTCCCGATGTTCGGAACTGCAAAGTACAGCCAAAACTTCCAGGGCCGGGTGGCCATCACAGCTGACGAGT
CAACCGGAACCGCCTCCATGGAGCTCTCTTCGCTCCGCTCGGAAGATACTGCCGTGTATTACTGCGCCCGGTCACG
GGACCCCCTCCTGTACCCACACCATGCTCTGTCCCCCTGGGGCAGAGGGACCATGGTCACTGTGTCCTCGGCATCC
ACTAAGGGGCCTAGCGTCTTTCCGCTGGCCCCGTCCTCCAAGTCCACTTCGGGTGGAACCGCGGCACTGGGGTGC
CTCGTGAAGGACTACTTCCCCGAGCCGGTCACCGTGTCCTGGAACTCGGGAGCCCTGACCTCCGGAGTGCATACTT
TCCCTGCGGTGCTGCAGTCCTCCGGGCTCTACTCGCTGTCAAGCGTGGTCACCGTCCCGAGCTCATCCCTGGGTAC
TCAGACCTACATTTGCAACGTGAACCACAAACCTTCCAACACCAAGGTCGACAAGAAAGTGGAGCCTAAGAGCTG
CGACAAGACCCACACCTGTCCCCCGTGTCCCGCCCCTGAGCTGCTGGGCGGCCCCAGCGTGTTCCTCTTCCCGCCT
AAGCCGAAGGACACTCTGTACATCACCAGAGAGCCTGAAGTGACCTGTGTGGTGGTGGATGTGTCCCACGAGGAT
CCGGAAGTGAAGTTCAATTGGTACGTGGACGGAGTGGAAGTCCATAACGCCAAGACCAAGCCCCGCGAGGAACA
GTACAACTCAACTTACCGGGTGGTGTCAGTGCTGACCGTGCTGCACCAAGATTGGCTGAACGGGAAGGAGTACAA
GTGCAAAGTCTCCAACAAGGCGCTGCCGGCCCCCATTGAAAAGACCATCAGCAAGGCTAAGGGCCAGCCCCGGG
AACCACAGGTCTACACCTTGCCCCCTTCCCGGGACGAACTGACCAAGAACCAAGTGTCGCTGACGTGCCTGGTCAA
GGGCTTTTATCCATCTGACATCGCCGTGGAGTGGGAAAGCAACGGCCAGCCGGAAAACAACTACAAGACTACCCC
GCCTGTGCTGGACTCCGACGGCTCGTTCTTCCTGTATTCCAAGCTCACCGTGGATAAGTCCAGATGGCAGCAGGGC
AATGTGTTCAGCTGCAGCGTGATGCATGAGGCCCTGCACAACCACTACACTCAGAAATCACTGTCCCTTTCCCCCG
GAAAA
SEQ ID NO: 12: Ab363A Nucleotide sequence of Light chain
TCGTCCGAACTGACCCAGGACCCGGCTGTGTCCGTGGCCCTGGGACAAACTGTGCGGGTCACGTGCCAGGGCGAC
AGCCTGCGCTACTACTACGCGTCGTGGTACCAGCAAAAGCCTGGGCAGGCGCCGGTGTTGGTGATCTACGGAAAG
AACAATCGCCCCTCCGGAATTCCAGACAGGTTCAGCGGCAGCTCATCCGGGAACACCGCCTCACTGACCATCACCG
GAGCCCAGGCCGAAGATGAAGCTGACTACTACTGCTCCTCGAGAGATTCCAGCGGCAACAACTGGGTGTTTGGTG
GCGGAACAGAGCTCACTGTGCTCGGGCAGCCGAAGGCTGCGCCCTCGGTGACCCTTTTCCCGCCATCATCCGAGG
AGCTGCAGGCCAACAAGGCCACGCTCGTGTGCCTGATCTCGGATTTCTACCCTGGTGCCGTGACTGTGGCCTGGA
AGGCAGACTCTAGCCCCGTGAAGGCCGGAGTGGAGACTACCACCCCGTCCAAGCAGTCCAACAACAAATACGCCG
CGTCATCCTACCTGTCCCTGACCCCTGAACAGTGGAAGTCCCATCGGAGCTATAGCTGCCAAGTCACCCACGAGGG
CTCCACCGTGGAAAAGACTGTCGCCCCCACCGAATGTTCG
SEQ ID NO: 13: Ab389A Nucleotide sequence of Heavy chain
CAAGTACAGTTGCAGCAGTCCGGAGCGGAAGTCAAGAAGCCGGGCTCCAGCGTGCGCGTGTCCTGTAAAGCCAG
CGGCGGCACCTTCAACAACGATGCCATTAACTGGGTCCGCCAGGCCCCGGGACAGGGTCTTGAATGGATGGGCG
GGATCTTCCCGCTTTTCGGAACTGCAAAGTACAGCCAAAACTTCCAGGGCCGGGTGGCCATCACAGCTGACGAGT
CAACCGGAACCGCCTCCATGGAGCTCTCTTCGCTCCGCTCGGAAGATACTGCCGTGTATTACTGCGCCCGGTCACG
GGACTTGCTCCTGTTCCCACACCATGCTCTGTCCCCCTGGGGCAGAGGGACCATGGTCACTGTGTCCTCGGCATCC
ACTAAGGGGCCTAGCGTCTTTCCGCTGGCCCCGTCCTCCAAGTCCACTTCGGGTGGAACCGCGGCACTGGGGTGC
CTCGTGAAGGACTACTTCCCCGAGCCGGTCACCGTGTCCTGGAACTCGGGAGCCCTGACCTCCGGAGTGCATACTT
TCCCTGCGGTGCTGCAGTCCTCCGGGCTCTACTCGCTGTCAAGCGTGGTCACCGTCCCGAGCTCATCCCTGGGTAC
TCAGACCTACATTTGCAACGTGAACCACAAACCTTCCAACACCAAGGTCGACAAGAAAGTGGAGCCTAAGAGCTG
CGACAAGACCCACACCTGTCCCCCGTGTCCCGCCCCTGAGCTGCTGGGCGGCCCCAGCGTGTTCCTCTTCCCGCCT
AAGCCGAAGGACACTCTGTACATCACCAGAGAGCCTGAAGTGACCTGTGTGGTGGTGGATGTGTCCCACGAGGAT
CCGGAAGTGAAGTTCAATTGGTACGTGGACGGAGTGGAAGTCCATAACGCCAAGACCAAGCCCCGCGAGGAACA
GTACAACTCAACTTACCGGGTGGTGTCAGTGCTGACCGTGCTGCACCAAGATTGGCTGAACGGGAAGGAGTACAA
GTGCAAAGTCTCCAACAAGGCGCTGCCGGCCCCCATTGAAAAGACCATCAGCAAGGCTAAGGGCCAGCCCCGGG
AACCACAGGTCTACACCTTGCCCCCTTCCCGGGACGAACTGACCAAGAACCAAGTGTCGCTGACGTGCCTGGTCAA
GGGCTTTTATCCATCTGACATCGCCGTGGAGTGGGAAAGCAACGGCCAGCCGGAAAACAACTACAAGACTACCCC
GCCTGTGCTGGACTCCGACGGCTCGTTCTTCCTGTATTCCAAGCTCACCGTGGATAAGTCCAGATGGCAGCAGGGC
AATGTGTTCAGCTGCAGCGTGATGCATGAGGCCCTGCACAACCACTACACTCAGAAATCACTGTCCCTTTCCCCCG
GAAAA
SEQ ID NO: 14: Ab389A Nucleotide sequence of Light chain
TCGTCCGAACTGACCCAGGACCCGGCTGTGTCCGTGGCCCTGGGACAAACTGTGCGGATCACGTGCCAGGGCGAC
TTCCTGCGCTCCAACTACGCGTCGTGGTACCAGCAAAAGCCTGGGCAGGCGCCGGTGTTGGTGATCTACGGAAAG
AACAATCGCCCCTCCGGAATTCCAGACAGGTTCAGCGGCAGCTCATCCGGGAACACCGCCTCACTGACCATCACCG
GAGCCCAGGCCGAAGATGAAGCTGACTACTACTGCTCCTCGAGAGATTCCAGCGGCAACCACTGGGTGTTTGGTG
GCGGAACAGAGCTCACTGTGCTCGGGCAGCCGAAGGCTGCGCCCTCGGTGACCCTTTTCCCGCCATCATCCGAGG
AGCTGCAGGCCAACAAGGCCACGCTCGTGTGCCTGATCTCGGATTTCTACCCTGGTGCCGTGACTGTGGCCTGGA
AGGCAGACTCTAGCCCCGTGAAGGCCGGAGTGGAGACTACCACCCCGTCCAAGCAGTCCAACAACAAATACGCCG
CGTCATCCTACCTGTCCCTGACCCCTGAACAGTGGAAGTCCCATCGGAGCTATAGCTGCCAAGTCACCCACGAGGG
CTCCACCGTGGAAAAGACTGTCGCCCCCACCGAATGTTCG
SEQ ID NO: 15: Ab389A CDRH1
NDAIN
SEQ ID NO: 16: Ab389A CDRH2
GIFPLFGTAKYSQNFQG
SEQ ID NO: 17: Ab389A CDRH3
SRDLLLFPHHALSP
SEQ ID NO: 18: Ab389A CDRL1
QGDFLRSNYAS
SEQ ID NO: 19: Ab389A CDRL2
GKNNRPS
SEQ ID NO: 20: Ab389A CDRL3
SSRDSSGNHWV
SEQ ID NO: 21: Ab389A Variable heavy chain
QVQLQQSGAEVKKPGSSVRVSCKASGGTFNNDAINWVRQAPGQGLEWMGGIFPLFGTAKYSQNFQGRVAITADEST
GTASMELSSLRSEDTAVYYCARSRDLLLFPHHALSPWGRGTMVTVSS
SEQ ID NO: 22: Ab389A Variable light chain
SSELTQDPAVSVALGQTVRITCQGDFLRSNYASWYQQKPGQAPVLVIYGKNNRPSGIPDRFSGSSSGNTASLTITGAQ
AEDEADYYCSSRDSSGNHWVFGGGTELTVL
SEQ ID NO: 23: Ab389A Heavy chain with YTE
QVQLQQSGAEVKKPGSSVRVSCKASGGTFNNDAINWVRQAPGQGLEWMGGIFPLFGTAKY
SQNFQGRVAITADESTGTASMELSSLRSEDTAVYYCARSRDLLLFPHHALSPWGRGTMVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPEL
LGGPSVFLFPPKPKDTLYITREPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO: 24: Ab389A Light chain
SSELTQDPAVSVALGQTVRITCQGDFLRSNYASWYQQKPGQAPVLVIYGKNNRPSGIPDR
FSGSSSGNTASLTITGAQAEDEADYYCSSRDSSGNHWVFGGGTELTVLGQPKAAPSVTLF
PPSSEELQANKATLVCLISDFYPGAVTVAWKADSSPVKAGVETTTPSKQSNNKYAASSYL
SLTPEQWKSHRSYSCQVTHEGSTVEKTVAPTECS

1-29. (canceled)

30. A B lymphocyte stimulator (BLyS) binding protein comprising the following 6 complementarity-determining regions (CDRs): CDRH1 of SEQ ID NO:1, CDRH2 of SEQ ID NO:2, CDRH3 of SEQ ID NO:3, CDRL1 of SEQ ID NO:4, CDRL2 of SEQ ID NO: 5, and CDRL3 of SEQ ID NO:6.

31. The BLyS binding protein according to claim 30, wherein the BLyS binding protein comprises a variable heavy (VH) region at least 90% identical or at least 95% identical to the sequence of SEQ ID NO:7 and/or a variable light (VL) region at least 90% identical or at least 95% identical to the sequence of SEQ ID NO:8.

32. The BLyS binding protein according to claim 30, wherein the BLyS binding protein comprises a variable heavy (VH) region of SEQ ID NO:7 and a variable light (VL) region of SEQ ID NO:8.

33. The BLyS binding protein according to claim 30, wherein the BLyS binding protein comprises an Fc (fragment crystallizable) region.

34. The BLyS binding protein according to claim 33, wherein the BLyS binding protein is an antibody.

35. The BLyS binding protein according to claim 34, wherein the antibody is a monoclonal antibody.

36. The BLyS binding protein according to claim 34, wherein the antibody is an IgG1 antibody.

37. The BLyS binding protein according to claim 35, wherein the IgG1 antibody is an IgG1 monoclonal antibody.

38. The BLyS binding protein according to claim 33, wherein the Fc region confers an increased half-life relative to a wild-type Fc region.

39. The BLyS binding protein according to claim 33, wherein the BLyS binding protein comprises a heavy chain Fc domain having a tyrosine residue at position 252, a threonine residue at position 254, and a glutamic acid residue at position 256 (according to EU index numbering).

40. The BLyS binding protein according to claim 30, wherein the BLyS binding protein comprises a heavy chain at least 90% identical or at least 95% identical to the sequence of SEQ ID NO:9 and a light chain at least 90% identical or at least 95% identical to the sequence of SEQ ID NO:10.

41. The BLyS binding protein according to claim 30, wherein the BLyS binding protein comprises a heavy chain of SEQ ID NO:9 and a light chain of SEQ ID NO:10.

42. A nucleic acid sequence which encodes a BLyS binding protein comprising:
(a) the following 6 complementarity-determining regions (CDRs): CDRH1 of SEQ ID NO: 1, CDRH2 of SEQ ID NO:2, CDRH3 of SEQ ID NO:3, CDRL1 of SEQ ID NO:4, CDRL2 of SEQ ID NO:5, and CDRL3 of SEQ ID NO:6;
(b) one or both of a variable heavy (VH) region of SEQ ID NO:7 and/or a variable light (VL) region of SEQ ID NO:8; and/or
(c) one or both of a heavy chain of SEQ ID NO:9 and/or a light chain of SEQ ID NO: 10.

43. The nucleic acid sequence according to claim 42, wherein the nucleic acid sequence comprises SEQ ID NO:11 encoding the heavy chain and/or SEQ ID NO: 12 encoding the light chain.

44. An expression vector comprising the nucleic acid sequence as defined in claim 42.

45. A recombinant host cell comprising the nucleic acid sequence as defined in claim 42.

46. A recombinant host cell comprising the expression vector as defined in claim 44.

47. A method for the production of a BLyS binding protein, wherein the method comprises culturing the recombinant host cell as defined in claim 45 under conditions suitable for expression of said nucleic acid sequence, whereby a polypeptide comprising the BLyS binding protein is produced.

48. The BLyS binding protein produced by the method of claim 47.

49. A cell line engineered to express the BLyS binding protein of claim 30.

50. A pharmaceutical composition comprising the BLyS binding protein as defined in claim 30 and a pharmaceutically acceptable excipient.

51. The pharmaceutical composition according to claim 50, wherein the BLyS binding protein comprises:
(a) a VH region at least 90% identical or at least 95% identical to the sequence of SEQ ID NO:7 and/or a VL region at least 90% identical or at least 95% identical to the sequence of SEQ ID NO:8;
(b) a VH region of SEQ ID NO:7 and a VL region of SEQ ID NO:8;
(c) a heavy chain at least 90% identical or at least 95% identical to the sequence of SEQ ID NO:9 and a light chain at least 90% identical or at least 95% identical to the sequence of SEQ ID NO:10; and/or
(d) a heavy chain of SEQ ID NO:9 and a light chain of SEQ ID NO:10.

52. A method for the treatment of a disease or condition in a human in need thereof comprising administering a therapeutically effective amount of (a) the BLyS binding protein as defined in claim 30 or (b) a pharmaceutical composition comprising the BLyS binding protein of claim 30 and a pharmaceutically acceptable excipient.

53. The method of treatment according to claim 52, wherein the BLyS binding protein comprises:
(a) a VH region at least 90% identical or at least 95% identical to the sequence of SEQ ID NO:7 and/or a VL region at least 90% identical or at least 95% identical to the sequence of SEQ ID NO:8;
(b) a VH region of SEQ ID NO:7 and a VL region of SEQ ID NO:8;
(c) a heavy chain at least 90% identical or at least 95% identical to the sequence of SEQ ID NO:9 and a light chain at least 90% identical or at least 95% identical to the sequence of SEQ ID NO:10; and/or
(d) a heavy chain of SEQ ID NO:9 and a light chain of SEQ ID NO:10.

54. The method of treatment according to claim 52, wherein the disease or condition is an autoimmune disease and/or a chronic inflammatory disease.

55. The method of treatment according to claim 52, wherein the disease or condition is systemic lupus erythematosus, lupus nephritis, systemic sclerosis, cutaneous lupus erythematosus, anti-neutrophil cytoplasmic antibody (ANCA)-associated vasculitis, systemic sclerosis-associated interstitial lung disease, connective tissue disease, and/or connective tissue disease-associated interstitial lung disease.

56. The method of treatment according to claim 52, wherein the disease or condition is systemic lupus erythematosus.

57. The method of treatment according to claim 52, wherein the disease or condition is lupus nephritis.

58. The method of treatment according to claim 52, wherein the disease or condition is connective tissue disease-associated interstitial lung disease.

59. A kit comprising:
(a) the BLyS binding protein as defined in claim 30; and
(b) instructions for use.
